SEQUENCE LISTING (24 sequences)

SEQ ID NO: 1 - protein, 516 aa, Thermoascus crustaceus
MRSFGPLALSLLGAAAVASAADAESDVRSLKKDTFDDFIKEHDLVLAEFFAPWCGHCQALAPEYEAAASELKEKNIPLAKVDCTEEADLCKEHGVEGYPTLKVFRGLDSVKPYPGARKSAAIVSYMVKQSLPAVSTVTKENLEEFKTMDKVVIIGYIAPDDKTSNETFTAFADSKRDDYLFAATNDAALAKAEGVKQPSIVLYKDFDEKKDVFDGKFDQETMLSWVKTASTPLVGEIGPETYAGYMSAGIPLAFIFAETPEERQKLAADFKPIAEKHRGAINIVTIDAKAFGAYAGNLNLDPQKFPCFAIHDTVKNAKFPYDQSKEVNAKDIGQFVQEVLDGKVEPSIKSEPIPETQEGSVTVVVAHSYRELVIDNDKDVLLEFYAPWCGHCKALAPKYEELGRLYAENPDLASKVTVAKIDATANDVPDEIQGFPTIKLFPAGAKDSPVDYAGSRTVEDLANFIKENGKHKVDALSAATAKPEQGGDVTSTPSISSETTAETAAPTTDGSEHDEL

SEQ ID NO: 2 - protein, 513 aa, Keithomyces carneus
MHHKRIAFGLLAAFASLASAEDTSDVHQLTEKTFDDFVKANPLVLAEFFAPWCGHCKALAPEYEEAATTLKEKNIKLAKIDCTEEADLCQKHGVEGYPTLKVFRGLDSVTPYNGQRKAAGITSYMVKQSLPAVSVLTKDTLEEFKTADKVVVVAYIAADDKASNETFTGVADKLRDNFLFGGVNDAAVAEAEGVKFPSIVLYKSFDEGKNTFTEKFEAEAIEKFTKTSATPLIGEVGPETYADYMSAGIPLAYIFAETQEERDSLSKDLKPVAEKYKGKINFATIDAKAFGAHAGNLNLQTDKFPAFAIHETVKNQKFPFDQEKKITKDAIAKFADEYSAGKIEPSIKSEPIPENQDGPVSIIVAKNYEQIVLDDTKDVLVEFYAPWCGHCKALAPKYDQLGEAYKNSEFKDKVVIAKVDATANDVPDEVSGFPTIKLFPAGKKSEPVTYNGARTVEDLIEFIKENGKYKAAVPFKEEATEEAAPAATEEKAEDKVEEEKKEEKKEDEDHDEL

SEQ ID NO: 3 - protein, 516 aa, Aspergillus spinulosporus
MRSFGPWALSLLGAAAAVSAADSQSETPSDVISLTKETFNDFLAEHDLVLAEFFAPWCGHCKALAPQYEEAATELKAKNIALVKVDCTAEEDVCREQEVTGYPTLKVFRGPDNVKPYQGARKTEAIVSYMVKQSLPAVSTVTAETLEDFKTMDKIVVVGYFAEDDKESSEAYTAFAESQRDNYLFASTNDAAVASAENVKQPSIVLYKDFDEKKAVYDGSLDSEALLSWVKTASTPLVGEVGPETYSGYIAAGIPLAYIFAETQEERTKFAEEFKPIAEKHRGAINIATIDAKAFGAHAGNLNLDPKTFPAFAIQDPAKNAKYPYDQTKEISAKDVGKFIQDVLEGKVEPSIKSEPVPETQEGPVTVVVAHSYKDLVIENDKDVLLEFYAPWCGHCKALAPKYDELAELYAKNEDFASKVTIAKIDATANDVPDSITGFPTIKLFPAGSKDAPVEYSGSRTVEDLANFVKENGKYGVDAFAAQSEEAEEAEEVAEDATETASDAEPSAEKPEHDEL

SEQ ID NO: 4 - protein, 517 aa, Thermoascus aurantiacus
MRSFGPLALSLLGAAAVASAANAESDVVSLKKDTFEDFIKEHDLVLAEFFAPWCGHCQALAPEYEAAASELKAKNIPLVKVDCTEEADLCKDYGVEGYPTLKVFRGLDSIKPYQGARKSGAIVSYMVKQSLPAVSTVTKENLEEFKTMDKVVIIGYIAAEDKASNETFTALAESKRDDYLFAVTSDPTLAKTEGVKQPSIVLYKDFDEKKDVFDGKFDKEAIISWIKTASTPLVGEIGPETYAGYMSAGIPLAFIFAETQEERQKFAADFKPIAEKHRGAINIVTIDAKAFGAYAGNLNLDAQKFPAFAIHDTVKNAKYPYDQSKEVNAKDIAQFVQDVLDGKVEPSIKSEPIPETQEGPVTVVVAHSYQELVIDNDKDVLLEFYAPWCGHCKALAPKYEELARLYAENPDLASKVTIAKIDATANDVPDEIQGFPTIKLFPAGSKDSPIDYTGSRTVEDLANFVKENGKHKVDALSAGTKKPQQGGDVTSAPSISSEKTAETVAPTRDGPVDHDEL

SEQ ID NO: 5 - protein, 110 aa, Aspergillus aculeatus
MSDHGKVHQITSTAEFREKVTNSQDAIVLDCFATWCGPCKAIAPKVAAFSEQYPQAKFYLIDVDELSDVAAELGVRAMPTFMLFKGGEKVKDVVGANPPALEAGIKALLA

SEQ ID NO: 6 - DNA, 1732 bp, Thermoascus crustaceus
  exon (1)..(362)
  exon (419)..(797)
  exon (868)..(1307)
  exon (1363)..(1732)
atgcggtctttcggacccctggctctcagcctgctcggtgctgcagcagtcgcctccgctgccgacgcagagtccgacgtccgctcgctcaagaaagatacgttcgatgatttcatcaaggagcatgaccttgttctagcagagttcttcgcgccttggtgtggtcattgtcaggcccttgcccccgaatatgaggctgctgcctccgagttaaaggagaagaacattcctctagctaaggtcgactgcacggaggaggcggatctgtgcaaggagcatggcgttgagggttatcccactctcaaggtcttccgtggccttgactcggtcaagccgtacccaggagctcggaagtccgcagcgtaagtgattgtgctggatgtggcagaattcatgagctgattaactctctcactagcattgtttcatacatggttaagcagtctctgccggccgtgtccaccgtgaccaaggagaatctcgaggagttcaagacgatggataaggtagtcatcatcggatacatcgctcctgatgacaaaacttccaacgagacgttcacggctttcgctgattcgaagcgggatgactacctttttgccgccactaacgacgctgctctggcaaaggcagaaggagttaagcagccttctattgtgctctacaaggattttgatgagaaaaaggatgtttttgacggaaaattcgaccaagagacgatgctcagctgggtaaaaacagctagcacccctcttgttggtgagatcggtccggagacctatgcgggatacatgtcggtaagtatacataacactgcgctgtctcttgttgcagtagttgcgagaagctaatacgaatcgaacctaggctggaattcccttggcgtttatctttgcggagacaccggaggagcgtcagaagctcgctgccgacttcaagcccatcgcggagaagcacaggggtgctatcaacattgtcacgattgatgctaaggcgtttggcgcttacgccggcaacctgaaccttgacccccaaaagtttccctgcttcgccatccatgacaccgtgaagaacgccaagttcccttacgatcagtccaaggaagtaaacgcgaaggatatcggccagtttgtccaggaagtccttgatggcaaggtcgagcctagcatcaagtccgagccaatccctgaaacccaagagggctctgttaccgttgtggtcgcccatagctaccgggagctggtgattgacaatgacaaggatgttctccttgagttctatgctccatggtgcggacactgcaaagcgtaagtcttaccaagcccttcaactttgagtaggtcatgtctgacggttgcttagtctcgctccgaagtacgaggagcttggacgcctttatgcggagaatcctgacctcgcatccaaggtcaccgttgccaaaatcgatgcgacagctaatgatgttcccgatgagattcaaggctttcccacaatcaagctcttccctgctggcgccaaggactccccggttgactacgctggatccagaactgtcgaggatctcgcgaactttatcaaagagaacggcaagcacaaggtggatgctcttagtgctgctactgcaaagcccgagcagggtggtgatgttactagtactccatcgatctcttcagaaacgaccgctgagactgctgccccgacaaccgatggatctgagcacgatgagctgtaa

SEQ ID NO: 7 - DNA, 1744 bp, Thermoascus aurantiacus
  exon (1)..(362)
  exon (427)..(805)
  exon (877)..(1316)
  exon (1372)..(1744)
atgcggtctttcggacccttggctttaagcctccttggcgccgctgcggtcgcctcagctgccaacgcagagtcggacgtcgtttcgctcaagaaggatacgttcgaagattttataaaggagcatgacctcgtgctggcggagttcttcgctccttggtgtggtcattgccaggccctcgcccccgaatatgaggctgccgcctcggagttgaaggcgaagaacattcctctggtaaaggttgactgtacggaagaggcggatctgtgcaaggactatggtgttgagggttatcccaccctcaaggtcttccgtggccttgactcgattaagccataccaaggagctcgaaagtccggagcgtaagccattttgtcactcccactggacatggcacaattcactggctgactaacattatcttaggatcgtttcgtatatggttaagcagtctctgccggcagtgtccacagtgaccaaggagaatcttgaggagttcaagaccatggacaaggtcgtcatcattggatacattgctgctgaagataaagcttccaacgagactttcacagctctcgcggagtcgaaacgggatgactacctttttgctgttaccagcgaccctacgttagcaaagacagaaggtgttaagcagccttctattgtcctctacaaggatttcgacgagaaaaaggatgtcttcgatggaaaattcgacaaagaggcaattattagctggattaaaacagccagtacacctctcgttggcgagattggcccggagacctatgcaggatatatgtcggtatgtatacatattacttcgttttttttgtctcaaaaagttgccgctaacatatgtaaaattcaatataggccggaattcctctggcgtttatctttgcggaaacgcaggaggagcggcaaaagtttgccgccgacttcaagcctattgcggagaagcacaggggtgctatcaacattgtcactattgacgctaaagcgttcggcgcttatgccgggaatctgaatcttgatgctcagaaattcccggccttcgccatccatgacaccgtgaagaacgctaagtacccttacgatcagtccaaggaagtaaatgcgaaggatattgcccagtttgtgcaagatgtcctagatggcaaggtcgagcctagcatcaagtccgaaccgattcctgaaacccaagagggtccagttaccgtcgtagtcgcccatagctaccaggagctagtgattgataacgacaaggatgtcctcctcgagttctatgccccgtggtgcgggcactgcaaagcgtaagttttaccaagccctccaacgtttagtgggaaatatctgaggtttggctagtctcgctccaaagtacgaggagctcgcacgcctttatgcggagaaccctgacctagcgtccaaggtcaccattgccaaaatcgatgcgacggcaaacgatgtacccgatgagattcaaggcttccccacgataaagctcttccctgccggctctaaggactctccgattgactacaccggatccagaactgtggaggaccttgcaaacttcgttaaggagaacgggaagcacaaggtagacgctcttagcgctggtactaaaaagccccagcagggcggtgatgttactagtgctccatcgatttcttcggaaaaaaccgccgagactgtcgccccgacaagagatggccctgtggatcatgatgagctgtaa

SEQ ID NO: 8 - protein, 551 aa, Artificial
  Synthetic construct
MDEKNLLEDPDSNLSAGASARALAATPMLHVGGRYLKDPCDNNVLLHGVAITPSPWFNGCQYGANSGYCTWDNYNVQGCLNYNKAVMNKLTSAADGWYLNYIRLHIDPYWTNDPGPSIPENDISRFNYNRLVTYTDQVIIPLINHARSRGMYVILRPPGVCPNRIAVNDAYHSYLKTVWTFLSQHPGLKNADNVMFELANEPVEILGTNGTWGQTGNEHFAALKNFFQPLVNIIRNNGANNVCWIPGTGWQSHYQGYPNNQITGGNIGYAVHIYPSYWGGLSNYQAFQNAWNINVKPIADIAPIAITETDWAPQGYGTFGIGTTGTAGGSGFGANLKYIVDQSGNVSWNVLAPDNLLHKGDPNGGTAYNNDWEACAAPVKQWFQQYAKSNYPVGNCNTTSSLVNNGIYEIEFQTDANKVVDLKSGEDANGAVLRPWTRNGAAAQRWVAIDAGNGYWRFVSKASATNRCITLASNSNTLGTSIVLWQNYGNDAQAWQVVAVSNGYYKILSKVDPTRGWDIPNCTMDGNSNLHLWDYYGTSCQLFKFKYIGMN

SEQ ID NO: 9 - protein, 551 aa, Chryseobacterium sp. 10696
MDEKNLLEDPDSNLSAGASARALAATPMLHVGGRYLKDPCDNNVVLHGVAITPSPWFNGCQYGANSGYCTWDNYNVQGALNYNKAVMNKLTSAADGWYLNYIRLHIDPYWTNDPGPAIPENDISRFNYNRLVTYTDQVIIPLINHARSLGMYVILRPPGVCPNRIAVNDAYHSYLKTVWTFLSQHPGLKNADNVMFELANEPVEILGTNGTWGSTGNEHFAALKNFFQPLVNIIRNNGANNVCWIPGTGWQSHYQGYVNNQITGGNIGYAVHIYPAYWGGLSNYQAFQNAWNINVKPIADIAPIAITETDWAPQGYGTFGIGTTGTAGGSGFGANLKYIVDQSGNVSWNVLAPDNLLHKGDPNAGTAYNNDWEACAAPVKQWFQQYASSNYPVGNCNTTSSLVNNGIYEIEFQTDANKVVDLKSGEDANGAVLRPWTRNGAAAQRWVAIDAGNGYWRFVSKASATNRCIDLASNSNTLGTSIRLWQNYGNDAQAWQVVAVSNGYYKILSKVDPTRGWDIPNCTMDGNSNLHLWDYYGTSCQLFKFKYIGMN

SEQ ID NO: 10 - protein, 319 aa, Aspergillus niger
MVQIKVAALAMLFASQVLSEPIEPRQASVSIDTKFKAHGKKYLGNIGDQYTLTKNSKTPAIIKADFGALTPENSMKWDATEPSRGQFSFSGSDYLVNFAQSNNKLIRGHTLVWHSQLPSWVQSITDKNTLIEVMENHITTVMQHYKGKIYAWDVVNEIFNEDGSLRDSVFYKVIGEDYVRIAFETARAADPNAKLYINDYNLDSASYPKLTGMVSHVKKWIAAGIPIDGIGSQTHLSAALNALAGAGTKEIAVTELDIAGASSTDYVEVVEACLNQPKCIGITVWGVADPDSWRSSSTPLLFDSNYNPKPAYTAIANAL

SEQ ID NO: 11 - protein, 405 aa, Talaromyces leycettanus
MVHLSSLALALAAGSQLAQAAGLNTAAKAIGKLYFGTATDNPELSDSTYMQETDNTDDFGQLTPANSMKWDATEPSQNTFTFTNGDQIANLAKSNGQMLRCHNLVWYNQLPSWVTSGSWTNATLLAAMKNHITNVVTHYKGQCYAWDVVNEALNDDGTYRSNVFYQYIGEAYIPIAFATAAAADPNAKLYYNDYNIEYPGAKATAAQNIVKMVKAYGAKIDGVGLQSHFIVGSTPSQSSQQSNMAAFTALGVEVAITELDIRMTLPSTSALLAQQSTDYQSTVSACVNTPKCIGITLWDWTDKYSWVPNTFSGQGDACPWDSNYQKKPAYYGILTALGGSASTSTTTTLVTSTRTSTTTSTSATSTSTGVAQHWGQCGGIGWTGPTTCASPYTCQELNPYYYQCL

SEQ ID NO: 12 - protein, 332 aa, Aspergillus luchuensis
MKFFNAKGSLLSSGIYLIALTPFVNAKCSLPSSYSWSSTDALATPKSGWTALKDFTDVVSDGKHIVYASTTDEAGNYGSMTFGAFSEWSNMASASQTATPFNAVAPTLFYFKPKSIWVLAYQWGSSTFTYRTSQDPTNVNGWSSEQALFTGKLSDSSTGAIDQTVIGDDTNMYLFFAGDNGKIYRSSMSIDEFPGSFGSQYEEILSGATNDLFEAVQVYTVDGGEGNSKYLMIVEAIGSTGHRYFRSFTASSLGGEWTAQAASEDKPFAGKANSGATWTEDISHGDLVRNNPDQTMTVDPCNLQLLYQGHDPNSSGDYNLLPWKPGVLTLKQ

SEQ ID NO: 13 - protein, 325 aa, Talaromyces pinophilus
MHFLAALLAVLPLVSGSPVPEKRSGCALPSTYKWTSTGPLASPKSGLVALRDYSHVIYNGQHLVYGSTANTAGSYGSMNFGLFSDWSEMSSASQNTMSTGAVAPTIFYFAPKSVWILAYQWGPYAFSYRTSTDPSNANGWSSPQPLFTGTISGSSTGVIDQTVIGDSENMYLFFAGDNGHIYRASMPIGDFPGSFGSASTIVLSDSTNNLFEAVEVYTVEGQNQYLMIVEAIGANGRYFRSFTASSLGGTWTAQASTESNPFAGKANSGATWTNDISSGDLVRTNPDQTQTIDACNLQFLYQGRSTSSGGDYNLLPYQPGLLTLA

SEQ ID NO: 14 - protein, 397 aa, Aspergillus fumigatus
MVHLSSLAAALAALPLVYGAGLNTAAKAKGLKYFGSATDNPELTDSAYVAQLSNTDDFGQITPGNSMKWDATEPSQNSFSFANGDAVVNLANKNGQLMRCHTLVWHSQLPNWVSSGSWTNATLLAAMKNHITNVVTHYKGKCYAWDVVNEALNEDGTFRNSVFYQIIGPAYIPIAFATAAAADPDVKLYYNDYNIEYSGAKATAAQNIVKMIKAYGAKIDGVGLQAHFIVGSTPSQSDLTTVLKGYTALGVEVAYTELDIRMQLPSTAAKLAQQSTDFQGVAAACVSTTGCVGVTIWDWTDKYSWVPSVFQGYGAPLPWDENYVKKPAYDGLMAGLGASGSGTTTTTTTTSTTTGGTDPTGVAQKWGQCGGIGWTGPTTCVSGTTCQKLNDWYSQCL

SEQ ID NO: 15 - protein, 792 aa, Aspergillus fumigatus
MAVAKSIAAVLVALLPGALAQANTSYVDYNVEANPDLTPQSVATIDLSFPDCENGPLSKTLVCDTSARPHDRAAALVSMFTFEELVNNTGNTSPGVPRLGLPPYQVWSEALHGLDRANFTNEGEYSWATSFPMPILTMSALNRTLINQIATIIATQGRAFNNVGRYGLDVYAPNINAFRSAMWGRGQETPGEDAYCLASAYAYEYITGIQGGVDPEHLKLVATAKHYAGYDLENWDGHSRLGNDMNITQQELSEYYTPQFLVAARDAKVHSVMCSYNAVNGVPSCANSFFLQTLLRDTFGFVEDGYVSSDCDSAYNVWNPHEFAANITGAAADSIRAGTDIDCGTTYQYYFGEAFDEQEVTRAEIERGVIRLYSNLVRLGYFDGNGSVYRDLTWNDVVTTDAWNISYEAAVEGIVLLKNDGTLPLAKSVRSVALIGPWMNVTTQLQGNYFGPAPYLISPLNAFQNSDFDVNYAFGTNISSHSTDGFSEALSAAKKSDVIIFAGGIDNTLEAEAMDRMNITWPGNQLQLIDQLSQLGKPLIVLQMGGGQVDSSSLKSNKNVNSLIWGGYPGQSGGQALLDIITGKRAPAGRLVVTQYPAEYATQFPATDMSLRPHGNNPGQTYMWYTGTPVYEFGHGLFYTTFHASLPGTGKDKTSFNIQDLLTQPHPGFANVEQMPLLNFTVTITNTGKVASDYTAMLFANTTAGPAPYPNKWLVGFDRLASLEPHRSQTMTIPVTIDSVARTDEAGNRVLYPGKYELALNNERSVVLQFVLTGREAVIFKWPVEQQQISSA

SEQ ID NO: 16 - protein, 532 aa, Aspergillus fumigatus
MLASTFSYRMYKTALILAALLGSGQAQQVGTSQAEVHPSMTWQSCTAGGSCTTNNGKVVIDANWRWVHKVGDYTNCYTGNTWDTTICPDDATCASNCALEGANYESTYGVTASGNSLRLNFVTTSQQKNIGSRLYMMKDDSTYEMFKLLNQEFTFDVDVSNLPCGLNGALYFVAMDADGGMSKYPTNKAGAKYGTGYCDSQCPRDLKFINGQANVEGWQPSSNDANAGTGNHGSCCAEMDIWEANSISTAFTPHPCDTPGQVMCTGDACGGTYSSDRYGGTCDPDGCDFNSFRQGNKTFYGPGMTVDTKSKFTVVTQFITDDGTSSGTLKEIKRFYVQNGKVIPNSESTWTGVSGNSITTEYCTAQKSLFQDQNVFEKHGGLEGMGAALAQGMVLVMSLWDDHSANMLWLDSNYPTTASSTTPGVARGTCDISSGVPADVEANHPDAYVVYSNIKVGPIGSTFNSGGSNPGGGTTTTTTTQPTTTTTTAGNPGGTGVAQHYGQCGGIGWTGPTTCASPYTCQKLNDYYSQCL

SEQ ID NO: 17 - protein, 454 aa, Aspergillus fumigatus
MKHLASSIALTLLLPAVQAQQTVWGQCGGQGWSGPTSCVAGAACSTLNPYYAQCIPGATATSTTLTTTTAATTTSQTTTKPTTTGPTTSAPTVTASGNPFSGYQLYANPYYSSEVHTLAMPSLPSSLQPKASAVAEVPSFVWLDVAAKVPTMGTYLADIQAKNKAGANPPIAGIFVVYDLPDRDCAALASNGEYSIANNGVANYKAYIDAIRAQLVKYSDVHTILVIEPDSLANLVTNLNVAKCANAQSAYLECVDYALKQLNLPNVAMYLDAGHAGWLGWPANLGPAATLFAKVYTDAGSPAAVRGLATNVANYNAWSLSTCPSYTQGDPNCDEKKYINAMAPLLKEAGFDAHFIMDTSRNGVQPTKQNAWGDWCNVIGTGFGVRPSTNTGDPLQDAFVWIKPGGESDGTSNSTSPRYDAHCGYSDALQPAPEAGTWFQAYFEQLLTNANPSF

SEQ ID NO: 18 - protein, 863 aa, Aspergillus fumigatus
MRFGWLEVAALTAASVANAQELAFSPPFYPSPWADGQGEWADAHRRAVEIVSQMTLAEKVNLTTGTGWEMDRCVGQTGSVPRLGINWGLCGQDSPLGIRDSDLNSAFPAGTNVAATWDKTLAYLRGKAMGEEFNDKGVDILLGPAAGPLGKYPDGGRIWEGFSPDPVLTGVLFAETIKGIQDAGVIATAKHYILNEQEHFRQVGEAQGYGYNITETISSNVDDKTMHELYLWPFADAVRAGVGAVMCSYNQINNSYGCQNSQTLNKLLKAELGFQGFVMSDWGAHHSGVGAALAGLDMSMPGDISFDDGLSFWGTNLTVSVLNGTVPAWRVDDMAVRIMTAYYKVGRDRLRIPPNFSSWTRDEYGWEHSAVSEGAWTKVNDFVNVQRSHSQIIREIGAASTVLLKNTGALPLTGKEVKVGVLGEDAGSNPWGANGCPDRGCDNGTLAMAWGSGTAEFPYLVTPEQAIQREVISNGGNVFAVTDNGALSQMADVASQSSVSLVFVNADSGEGYISVDGNEGDRKNLTLWKNGEAVIDTVVSHCNNTIVVIHSVGPVLIDRWYDNPNVTAIIWAGLPGQESGNSLVDVLYGRVNPSAKTPFTWGKTRESYGAPLLTEPNNGNGAPQDDFNEGVFIDYRHFDKRNETPIYEFGHGLSYTTFGYSHLRVQALNSSSSAYVPTSGETKPAPTYGEIGSAADYLYPEGLKRITKFIYPWLNSTDLEDSSDDPNYGWEDSEYIPEGARDGSPQPLLKAGGAPGGNPTLYQDLVRVSATITNTGNVAGYEVPQLYVSLGGPNEPRVVLRKFDRIFLAPGEQKVWTTTLNRRDLANWDVEAQDWVITKYPKKVHVGSSSRKLPLRAPLPRVY

SEQ ID NO: 19 - protein, 253 aa, Penicillium emersonii
MLSSTTRTLAFTGLAGLLSAPLVKAHGFVQGIVIGDQFYSGYIVNSFPYESNPPPVIGWATTATDLGFVDGTGYQGPDIICHRNATPAPLTAPVAAGGTVELQWTPWPDSHHGPVITYLAPCNGNCSTVDKTTLEFFKIDQQGLIDDTSPPGTWASDNLIANNNSWTVTIPNSVAPGNYVLRHEIIALHSANNKDGAQNYPQCINIEVTGGGSDAPEGTLGEDLYHDTDPGILVDIYEPIATYTIPGPPEPTF

SEQ ID NO: 20 - DNA, 1551 bp, Thermoascus crustaceus
atgcggtctttcggacccctggctctcagcctgctcggtgctgcagcagtcgcctccgctgccgacgcagagtccgacgtccgctcgctcaagaaagatacgttcgatgatttcatcaaggagcatgaccttgttctagcagagttcttcgcgccttggtgtggtcattgtcaggcccttgcccccgaatatgaggctgctgcctccgagttaaaggagaagaacattcctctagctaaggtcgactgcacggaggaggcggatctgtgcaaggagcatggcgttgagggttatcccactctcaaggtcttccgtggccttgactcggtcaagccgtacccaggagctcggaagtccgcagccattgtttcatacatggttaagcagtctctgccggccgtgtccaccgtgaccaaggagaatctcgaggagttcaagacgatggataaggtagtcatcatcggatacatcgctcctgatgacaaaacttccaacgagacgttcacggctttcgctgattcgaagcgggatgactacctttttgccgccactaacgacgctgctctggcaaaggcagaaggagttaagcagccttctattgtgctctacaaggattttgatgagaaaaaggatgtttttgacggaaaattcgaccaagagacgatgctcagctgggtaaaaacagctagcacccctcttgttggtgagatcggtccggagacctatgcgggatacatgtcggctggaattcccttggcgtttatctttgcggagacaccggaggagcgtcagaagctcgctgccgacttcaagcccatcgcggagaagcacaggggtgctatcaacattgtcacgattgatgctaaggcgtttggcgcttacgccggcaacctgaaccttgacccccaaaagtttccctgcttcgccatccatgacaccgtgaagaacgccaagttcccttacgatcagtccaaggaagtaaacgcgaaggatatcggccagtttgtccaggaagtccttgatggcaaggtcgagcctagcatcaagtccgagccaatccctgaaacccaagagggctctgttaccgttgtggtcgcccatagctaccgggagctggtgattgacaatgacaaggatgttctccttgagttctatgctccatggtgcggacactgcaaagctctcgctccgaagtacgaggagcttggacgcctttatgcggagaatcctgacctcgcatccaaggtcaccgttgccaaaatcgatgcgacagctaatgatgttcccgatgagattcaaggctttcccacaatcaagctcttccctgctggcgccaaggactccccggttgactacgctggatccagaactgtcgaggatctcgcgaactttatcaaagagaacggcaagcacaaggtggatgctcttagtgctgctactgcaaagcccgagcagggtggtgatgttactagtactccatcgatctcttcagaaacgaccgctgagactgctgccccgacaaccgatggatctgagcacgatgagctgtaa

SEQ ID NO: 21 - DNA, 1542 bp, Keithomyces carneus
atgcatcacaagcgtattgccttcggcctcttggccgcctttgcctctctggcctctgccgaggacacctccgacgttcaccagttgaccgagaagacttttgacgatttcgtcaaggccaaccccttggttctcgccgagttctttgctccctggtgcggacactgcaaggccctcgctcccgagtatgaagaggctgcaacaacgctcaaggagaagaacatcaagctggccaagatcgactgcaccgaagaggccgacctctgccagaagcacggcgtcgagggctaccccaccctcaaggtcttccgtggtcttgacagcgtcaccccttacaatggtcagcgcaaggctgctggtatcacctcatacatggtcaagcagtctctgcctgccgtctctgtcctgaccaaggacacccttgaggagttcaagactgccgacaaggttgttgttgttgcctacatcgctgccgatgacaaggcctccaatgagaccttcacaggtgttgctgataagctccgcgacaacttcctgtttggtggtgtcaacgatgccgccgtcgctgaggccgagggcgtcaagttcccctccattgttctgtacaagtctttcgacgagggcaagaacaccttcacagagaagttcgaggccgaggccatcgagaagttcaccaagacttccgccacccctctgattggcgaggtcggccccgagacctacgctgactacatgtctgccggcattcccctggcttacatcttcgccgagacccaggaggagcgtgacagcctctccaaggatctcaagcctgttgctgagaagtacaagggcaagatcaacttcgccaccattgacgcaaaggctttcggtgctcacgccggcaacctgaacctccagactgacaagttccctgcctttgccatccacgagaccgtcaagaaccagaagttccccttcgaccaggagaagaaaatcacaaaggacgccattgccaagtttgctgatgagtactcagctggcaagattgagcctagcatcaagtcggagcccattcctgagaaccaggacggtcccgtcagcatcattgtcgccaagaactacgaacagattgttcttgacgacaccaaggatgtccttgttgagttctacgccccctggtgcggtcactgcaaggctcttgctcccaagtacgaccagcttggtgaggcctacaagaactccgagttcaaggacaaggttgtcattgccaaggtcgatgctaccgctaacgatgtccctgatgaggtttctggtttccccaccatcaagctgttccccgctggcaagaagtccgagcctgtcacttacaacggcgctcgtactgtcgaggacctgatcgagttcatcaaggagaacggcaagtacaaggctgccgtccccttcaaggaggaggccactgaggaggctgcccccgccgctaccgaggagaaggctgaagacaaggtcgaggaggagaagaaggaggagaagaaggaagatgaggaccacgacgagctgtaa

SEQ ID NO: 22 - DNA, 1551 bp, Aspergillus spinulosporus
atgcgttccttcggaccgtgggcgctgagccttctaggggcagcagcagcagtttctgctgcggattcccagtccgaaacaccatcggacgtcatatcgttgaccaaggagactttcaatgatttcctggcggagcatgacctagtccttgcggagtttttcgcgccctggtgtggtcactgcaaagctctagcacctcaatacgaagaagcagctaccgagttgaaggccaaaaatatcgcattggtcaaggttgactgcactgcggaggaggatgtttgtagggagcaggaagtgaccggttaccctacccttaaggtcttccgcggccctgataatgtgaagccctatcaaggagctcggaagacagaagcaattgtctcgtacatggtgaaacaatcgctccctgcggtttccactgtgactgcggaaacccttgaggacttcaagaccatggacaagatcgttgttgtcggctatttcgctgaggatgataaggaatcgtctgaagcttacacggccttcgctgagagccagagggataactatttgtttgcttctaccaatgacgctgcggttgctagcgctgaaaatgtgaagcagccttccattgtgctctacaaagactttgatgagaaaaaagccgtctacgatggatcgctcgactctgaagccctgctcagctgggtgaagaccgccagcactcccctcgtgggcgaggttgggcctgagacctactctgggtacatcgcggccggcatccccctggcttacatattcgccgagacccaggaagagcgcactaagttcgctgaggagttcaaacccattgcagagaagcacaggggtgctatcaacatagccacaattgacgccaaggcttttggtgcccatgccggaaacctcaaccttgaccccaagacgttccctgcctttgccatccaggatcccgcgaagaacgcgaagtacccgtacgaccaaactaaggaaatctcagccaaggatgtcggcaagtttattcaggatgtcctagagggcaaggttgaacccagcatcaagtctgaacccgtgcctgaaactcaagaaggtcctgtcacagttgtggtggcccactcatacaaggatcttgtgatcgagaacgacaaggatgtccttcttgagttctacgcaccatggtgcggacattgcaaggcccttgcccccaaatatgatgaattggccgagttgtacgcgaagaacgaagatttcgcatctaaggtcactattgcaaagattgacgcgacggccaacgacgtccctgactctatcaccggtttcccgaccatcaaattattccctgctgggtccaaagatgcccctgtcgaatattcgggttctcgtaccgtggaggacctcgcaaacttcgtcaaagagaacggcaaatacggagttgatgctttcgccgctcagtccgaggaggccgaggaggccgaggaggttgcggaagatgctacagagaccgctagcgatgctgagccttccgctgaaaagcctgagcatgatgagctttga

SEQ ID NO: 23 - DNA, 1554 bp, Thermoascus aurantiacus
atgcggtctttcggacccttggctttaagcctccttggcgccgctgcggtcgcctcagctgccaacgcagagtcggacgtcgtttcgctcaagaaggatacgttcgaagattttataaaggagcatgacctcgtgctggcggagttcttcgctccttggtgtggtcattgccaggccctcgcccccgaatatgaggctgccgcctcggagttgaaggcgaagaacattcctctggtaaaggttgactgtacggaagaggcggatctgtgcaaggactatggtgttgagggttatcccaccctcaaggtcttccgtggccttgactcgattaagccataccaaggagctcgaaagtccggagcgatcgtttcgtatatggttaagcagtctctgccggcagtgtccacagtgaccaaggagaatcttgaggagttcaagaccatggacaaggtcgtcatcattggatacattgctgctgaagataaagcttccaacgagactttcacagctctcgcggagtcgaaacgggatgactacctttttgctgttaccagcgaccctacgttagcaaagacagaaggtgttaagcagccttctattgtcctctacaaggatttcgacgagaaaaaggatgtcttcgatggaaaattcgacaaagaggcaattattagctggattaaaacagccagtacacctctcgttggcgagattggcccggagacctatgcaggatatatgtcggccggaattcctctggcgtttatctttgcggaaacgcaggaggagcggcaaaagtttgccgccgacttcaagcctattgcggagaagcacaggggtgctatcaacattgtcactattgacgctaaagcgttcggcgcttatgccgggaatctgaatcttgatgctcagaaattcccggccttcgccatccatgacaccgtgaagaacgctaagtacccttacgatcagtccaaggaagtaaatgcgaaggatattgcccagtttgtgcaagatgtcctagatggcaaggtcgagcctagcatcaagtccgaaccgattcctgaaacccaagagggtccagttaccgtcgtagtcgcccatagctaccaggagctagtgattgataacgacaaggatgtcctcctcgagttctatgccccgtggtgcgggcactgcaaagctctcgctccaaagtacgaggagctcgcacgcctttatgcggagaaccctgacctagcgtccaaggtcaccattgccaaaatcgatgcgacggcaaacgatgtacccgatgagattcaaggcttccccacgataaagctcttccctgccggctctaaggactctccgattgactacaccggatccagaactgtggaggaccttgcaaacttcgttaaggagaacgggaagcacaaggtagacgctcttagcgctggtactaaaaagccccagcagggcggtgatgttactagtgctccatcgatttcttcggaaaaaaccgccgagactgtcgccccgacaagagatggccctgtggatcatgatgagctgtaa

SEQ ID NO: 24 - DNA, 333 bp, Aspergillus aculeatus
atgtctgaccacggcaaggttcaccagatcacttccactgcggagttcagagagaaggtcaccaactcccaggacgccattgtcctcgactgcttcgccacgtggtgcggtccctgcaaggccatcgcccccaaggtcgccgctttcagcgaacaatacccccaggccaagttctacttgatcgacgtcgacgagctctctgatgtcgctgccgaactcggtgtccgcgccatgcccaccttcatgctgttcaagggcggcgagaaggtcaaggacgttgtcggtgccaacccccccgctcttgaggccggtatcaaggctctcctcgcatag

(19) United States
(12) Patent Application Publication
(10) Pub. No.: US 2024/0307885 A1
(43) Pub. Date: Sep. 19, 2024

(54) IMPROVED FIBER-WASH IN CORN WET-MILLING

(71) Applicant: NOVOZYMES A/S, Bagsvaerd (DK)

(72) Inventors: Svend Gunnar Kaasgaard, Skovlunde (DK); Mary Ann Stringer, Soborg (DK); Madelyn Mallison Shoup, Raleigh, NC (US); Bemardo Vidal, Wake Forest, NC (US); Wei Peng, Shanghai (CN); Xiangyu Cai, Beijing (CN); Yi Cao, Shanghai (CN)

(73) Assignee: NOVOZYMES A/S, Bagsvaerd (DK)

(21) Appl. No.: 18/257,665

(22) PCT Filed: Dec. 17, 2021
(86) PCT No.: PCT/CN2021/139046

§ 371 Date: Jun. 15, 2023

(30) Foreign Application Priority Data
Dec. 17, 2020 (WO) PCT/CN2020/137119

(51) Int. Cl. B02B 5/02; B02B 1/04; C12N 9/02; C12N 9/90

(52) U.S. Cl. CPC B02B 5/02; B02B 1/04; C12N 9/0004; C12N 9/90; C12Y 503/04001

(57) ABSTRACT

The instant application provides methods to increase the total starch yield and/or gluten yield from corn kernels in a wet milling process, the method comprising admixing corn kernels or a fraction of the corn kernels with a Protein Disulfide Isomerase or Thioredoxin.

### FIELD OF THE INVENTION

The present invention relates to a method of improving/increasing starch and/or gluten yield from corn kernels in a wet milling process, by contacting said corn kernels with an enzyme composition, preferably during fiber washing.

### BACKGROUND OF THE INVENTION

Conventional wet milling of corn is a process designed for the recovery and purification of starch and several coproducts including germ, gluten (protein) and fiber. Fiber is the least valuable coproduct, so the industry has put substantial effort into increasing the yield of the more valuable products, such as starch and gluten, while decreasing the fiber fraction. High quality starch is valuable as it can be used for a variety of commercial purposes after further processing to products such as dried starch, modified starch, dextrins, sweeteners and alcohol. Gluten is usually used for animal feed, as corn gluten meal (Around 60% protein) or corn gluten feed (Around 20% protein).

The wet milling process can vary significantly dependent on the specific mill equipment used, but usually the process include: grain cleaning, steeping, grinding, germ separation, a second grinding, fiber separation, gluten separation and starch separation. After cleaning the corn kernels, they are typically softened by soaking in water or in a dilute SO₂ solution under controlled conditions of time and temperature. Then, the kernels are ground to break down the pericarp and the germ is separated from the rest of the kernel. The remaining slurry, mainly consisting of fiber, starch and gluten is finely ground and screened in a fiber washing process, to separate the fiber from starch and gluten, before the gluten and starch is separated and the starch can be purified in a washing/filtration process.

The use of enzymes in several steps of the wet milling process has been suggested, such as the use of enzymes for the steeping step of wet milling processes. The commercial enzyme product Steepzyme® (available from Novozymes A/S) has been shown suitable for the first step in wet milling processes, i.e., the steeping step where corn kernels are soaked in water.

More recently, “enzymatic milling”, a modified wet milling process that uses proteases to significantly reduce the total processing time during corn wet milling and eliminates the need for sulfur dioxide as a processing agent, has been developed. Johnston et al., Cereal Chem, 81, p. 626-632 (2004).
U.S. Pat. No. 6,566,125 discloses a method for obtaining starch from maize involving soaking maize kernels in water to produce soaked maize kernels, grinding the soaked maize kernels to produce a ground maize slurry, and incubating the ground maize slurry with enzyme (e.g., protease).
U.S. Pat. No. 5,066,218 discloses a method of milling grain, especially corn, comprising cleaning the grain, steeping the grain in water to soften it, and then milling the grain with a cellulase enzyme.
WO 2002/000731 discloses a process of treating crop kernels, comprising soaking the kernels in water for 1-12 hours, wet milling the soaked kernels and treating the kernels with one or more enzymes including an acidic protease.
WO 2002/000911 discloses a process of starch gluten separation, comprising subjecting mill starch to an acidic protease.
WO 2002/002644 discloses a process of washing a starch slurry obtained from the starch gluten separation step of a milling process, comprising washing the starch slurry with an aqueous solution comprising an effective amount of acidic protease.
WO 2014/082566 and WO 2014/082564 disclose cellulolytic compositions for use in wet milling.
WO2016/095856 discloses compositions comprising xylanases and arabinofuranosidases and use of these copositions in fiber-wash in a corn wet-milling process.
WO2019/023222 discloses a wet-milling process applying GH5 xylanases and GH30 xylanases in combination with cellulases in the fiber-washing step.
WO2017/088820 discloses a process for improved starch release in corn wet-milling from fiber by adding an alpha-L-arabinofuranosidase (GH62) alone or in combination with a xylanase (GH10) in a fiberwash step.
WO2018/053220 discloses a fiber-washing system as part of a wet-milling process optimized for applying enzymes in the fiber-washing step by using a dedicated space/tank for the enzyme incubation.

While the art has investigated the effect of using enzymes in corn wet milling, during steeping/soaking of corn kernels, during grinding of the corn kernels, and in starch gluten separation, there is still a need for improved technology that may lower the energy expenditure and costs associated with corn wet milling and provide increased yield of starch and gluten.

### SUMMARY OF THE INVENTION

In a first aspect the present invention relates to a method for increasing starch yield and/or gluten yield from corn kernels in a wet milling process, the method comprising contacting ground corn kernels or a fraction of the ground kernels, particularly a fiber rich fraction, with an effective amount of a polypeptide that catalyzes changes in protein disulfide bonds, including oxidation, reduction, or isomerization, such as a Thioredoxin peptide, or a Protein Disulfide Isomerase (PDI) (EC 5.3.4.1).

In further aspects the present invention relates to isolated polypeptides having protein disulfide activity or to thioredoxin peptides capable of catalyzing changes in protein disulfide bonds, including oxidation, reduction, or isomerization.

The present invention also relates to polynucleotides encoding the polypetides having protein disulfide activity or to thioredoxin peptides capable of catalyzing changes in protein disulfide bonds, including oxidation, reduction, or isomerization; nucleic acid constructs, vectors, and host cells comprising the polynucleotides; and methods of producing the polypeptides of the invention.

Furthermore, the invention relates to compositions comprising the polypeptides of the invention.

The present invention also relates to methods of producing the polypeptides of the invention.

### Definitions

### Definition of Enzymes:

Protein Disulfide Isomerase (PDI): The term Protein disulfide isomerase (herein also referred to as PDI) (EC 5.3.4.1), means an enzyme that catalyzes the formation and breakage of disulfide bonds between residues within proteins as they fold by way of oxidation, reduction, or isomerization. PDIs are members of the thioredoxin superfamily of redox proteins, and typically comprise at least three domains, two catalytic domains each comprising an active site characterized by having a CXXC, preferably a CGHC motif, separated by at least one non-catalytic domain (Perri et al., 2016, Front. Cell Dev.Biol. 3:80, Hatahet and Ruddock, 2007, FEBS Journal 274: 5223-5234). In one embodiment the active site motif is selected from CGHC, CTHC, CPHC, and CSMC. The catalytic domains typically belong to the protein domain family Pf00085 (thioredoxin, https://pfam.xfam.org/family/Thioredoxin), as classified by the Pfam database, a large collection of protein domain families (Mistry, et al., 2020, “Pfam: The protein families database in 2021”, Nucleic Acids Research, doi: 10.1093/nar/gkaa913; http://pfam.xfam.org,) The non-catalytic domain can belong to the Pfam domain Pf13848 (thioredoxin_6 domain, https://pfam.xfam.org/family/PF13848). In a preferred embodiment the PDI suitable for the process of the invention comprises at least one Pf00085 domain including an active site consisting of the polypeptide motif CGHC, CTHC, CPHC, or CSMC, preferably CGHC. In another preferred embodiment the PDI comprises a three domain structure with the domains arranged in in the sequential order Pf00085, Pf13848,Pf00085. The 3 domains may be separated by peptide linkers. PDI activity means that the polypeptide has activity in the Insulin reduction assay (using insulin as substrate and in the presence of DTT) described in the assay section in the examples pages 57-59 (Insulin reduction assay with dithiothreitol), and applied in Example 7.

Thioredoxinpeptide (Trx): The term thioredoxin means a protein that catalyzes the reduction of protein disulfide bonds by cysteine thiol-disulfide exchange. Thioredoxins act as antioxidants by facilitating the reduction of other proteins (Collet and Messens, 2010, Antioxid. Redox Signal. 13, 1205-1216). Thioredoxins are small oxidoreductase enzymes typically around 12-kD in size and contain a dithiol-disulfide active site, e.g., CGPC. They are ubiquitous and found in many organisms from plants and bacteria to mammals. Multiple in vitro substrates for thioredoxin have been identified, including ribonuclease, choriogonadotropins, coagulation factors, glucocorticoid receptor, and insulin. Reduction of insulin is classically used as an activity test. Thioredoxins are characterized at the level of their amino acid sequence by the presence of two vicinal cysteines in a CXXC motif. These two cysteines are the key to the ability of thioredoxin to reduce other proteins. Thioredoxin proteins also have a characteristic tertiary structure termed the thioredoxin fold, that is also found as a component of other proteins such as glutaredoxins, PDIs, and disulfide oxidases. Therefore, according to this invention a thioredoxin peptide is a member of the thioredoxin superfamily of redox proteins, and typically comprise a catalytic domain comprising an active site characterized by having a CXXC motif, preferably a CGPC motif.

Thioredoxin-like peptides comprise a domain that belongs to the protein domain family Pf00085 (thioredoxin, https://pfam.xfam.org/family/Thioredoxin) as classified by the Pfam database (http://pfan.xfan.orgl). Thioredoxin peptide activity means that the peptide comprising the Pf00085 domain has activity in the Insulin reduction assay described in the examples. A thioredoxin peptide according to the invention should have activity in the Insulin reduction assay (using insulin as substrate and in the presence of DTT) disclosed in the examples, e.g., Examples 7.

Arabinofuranosidases/polypeptide with arabinofuranosidase activity: The term “arabinofuranosidase” means an alpha L-arabinofuranoside arabinofuranohydrolase (EC 3.2.1.55) that catalyzes the hydrolysis of terminal non-reducing alpha-L-arabinofuranoside residues in alpha-L-arabinosides. The enzyme acts on alpha-L-arabinofuranosides, alpha-L-arabinans containing (1,3)— and/or (1,2)— and/or (1,5)-linkages, arabinoxylans, and arabinogalactans. Alpha-L arabinofuranosidase is also known as arabinosidase, alpha-arabinosidase, alpha-L-arabinosidase, alphaarabinofuranosidase,polysaccharide alpha-L-arabinofuranosidase, alpha-L-arabinofuranoside hydrolase, L-arabinosidase, or alpha-L-arabinanase. Arabinofuranosidase activity can be determined using 5 mg of medium viscosity wheat arabinoxylan (Megazyme International Ireland, Ltd., Bray, Co. Wicklow, Ireland) per ml of 100 mM sodium acetate pH 5 in a total volume of 200 μl for 30 minutes at 40° C. followed by arabinose analysis by AMINEX® HPX-87H column chromatography (Bio-Rad Laboratories, Inc., Hercules, CA, USA). Arabinofuranosidases can be found in, e.g., the GH43, GH62, GH51 families according to Henrissat, 1991, A classification of glycosyl hydrolases based on amino-acid sequence similarities, Biochem. J. 280: 309-316, and Henrissat and Bairoch, 1996, Updating the sequence-based classification of glycosyl hydrolases, Biochem. J. 316: 695-696.

Beta-glucosidase/polypeptide with beta-glucosidase activity: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. Beta-glucosidase activity can be determined using p-nitrophenyl-beta-D-glucopyranoside as substrate according to the procedure of Venturi et al.,2002, J. Basic Microbiol. 42: 55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN® 20.

Beta-xylosidase/polypeptide with beta-xylosidase activity: The term “beta-xylosidase” means a beta-D-xyloside xylohydrolase (E.C. 3.2.1.37) that catalyzes the exo-hydrolysis of short beta (1→4)-xylooligosaccharides to remove successive D-xylose residues from non-reducing termini. Beta-xylosidase activity can be determined using 1 mM p-nitrophenyl-beta-D-xyloside as substrate in 100 mM sodium citrate containing 0.01% TWEEN® 20 at pH 5, 40° C. One unit of beta-xylosidase is defined as 1.0 μmole of p-nitrophenolate anion produced per minute at 40° C., pH 5 from 1 mM p-nitrophenyl-beta-D-xyloside in 100 mM sodium citrate containing 0.01% TWEEN® 20.

Cellobiohydrolase/polypeptide with cellobiohydrolase activity: The term “cellobiohydrolase” means a 1,4-beta-D-glucan cellobiohydrolase (E.C. 3.2.1.91 and E.C. 3.2.1.176) that catalyzes the hydrolysis of 1,4-beta-D-glucosidic linkages in cellulose, cellooligosaccharides, or any beta-1,4-linked glucose containing polymer, releasing cellobiose from the reducing end (cellobiohydrolase 1) or non-reducing end (cellobiohydrolase 1l) of the chain (Teeri, 1997, Trends in Biotechnology 15: 160-167; Teeri et al., 1998, Biochem. Soc. Trans. 26: 173-178). Cellobiohydrolase activity can be determined according to the procedures described by Lever et al., 1972, Anal. Biochem. 47: 273-279; van Tilbeurgh et al., 1982, FEBS Letters 149: 152-156; van Tilbeurgh and Claeyssens, 1985, FEBS Letters 187: 283-288; and Tomme et al., 1988, Eur. J. Biochem. 170: 575-581.

Cellulolytic enzyme or cellulase/polypeptide with cellulase activity or cellulolytic activity: The term “cellulolytic enzyme” or “cellulase” means one or more (e.g., several) enzymes that hydrolyze a cellulosic material, which comprise any material comprising cellulose, such as fiber. Cellulytic enzymes include endoglucanase(s) (E.C 3.2.1.4), cellobiohydrolase(s) (E.C 3.2.1.91 and E.C 3.2.1.150), beta-glucosidase(s) (E.C. 3.2.1.21), or combinations thereof. The two basic approaches for measuring cellulolytic enzyme activity include: (1) measuring the total cellulolytic enzyme activity, and (2) measuring the individual cellulolytic enzyme activities (endoglucanases, cellobiohydrolases, and beta-glucosidases) as reviewed in Zhang et al., 2006, Biotechnology Advances 24: 452-481. Total cellulolytic enzyme activity can be measured using insoluble substrates, including Whatman No 1 filter paper, microcrystalline cellulose, bacterial cellulose, algal cellulose, cotton, pretreated lignocellulose, etc. The most common total cellulolytic activity assay is the filter paper assay using Whatman NW1 filter paper as the substrate. The assay was established by the International Union of Pure and Applied Chemistry (IUPAC) (Ghose, 1987, Pure Appl. Chem. 59: 257-68).

Cellulolytic enzyme activity can be determined by measuring the increase in production/release of sugars during hydrolysis of a cellulosic material by cellulolytic enzyme(s) under the following conditions: 1-50 mg of cellulolytic enzyme protein/g of cellulose in pretreated corn stover (PCS) (or other pretreated cellulosic material) for 3-7 days at a suitable temperature such as 40° C.-80° C., e.g., 40° C., 45° C., 50° C., 55° C., 60° C., 65° C., 70° C., 75° C., or 80° C., and a suitable pH, such as 4-9, e.g., 4.0, 4.5, 5.0, 5.5, 6.0, 6.5, 7.0, 7.5, 8.0, 8.5, or 9.0, compared to a control hydrolysis without addition of cellulolytic enzyme protein. Typical conditions are 1 ml reactions, washed or unwashed PCS, 5% insoluble solids (dry weight), 50 mM sodium acetate pH 5, 1 mM MnSO4, 50° C., 55° C., or 60° C., 72 hours, sugar analysis by AMINEX® HPX-87H column chromatography (Bio-Rad Laboratories, Inc., Hercules, CA, USA).

Endoglucanase: The term “endoglucanase” means an endo-1,4-(1,3;1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). For purposes of the present invention, endoglucanase activity is determined using carboxymethyl cellulose (CMC) as substrate according to the procedure of Ghose, 1987, Pure and Appl. Chem. 59: 257-268, at pH 5, 40° C.

Family 61 glycoside hydrolase: The term “Family 61 glycoside hydrolase” or “Family GH61” or “GH61” means a polypeptide falling into the glycoside hydrolase Family 61 according to Henrissat, 1991, A classification of glycosyl hydrolases based on amino-acid sequence similarities, Biochem. J. 280: 309-316, and Henrissat and Bairoch, 1996, Updating the sequence-based classification of glycosyl hydrolases, Biochem. J. 316: 695-696. The enzymes in this family were originally classified as a glycoside hydrolase family based on measurement of very weak endo-1,4-beta-D-glucanase activity in one family member. The structure and mode of action of these enzymes are non-canonical and they cannot be considered as bona fide glycosidases. However, they are kept in the CAZy classification on the basis of their capacity to enhance the breakdown of lignocellulose when used in conjunction with a cellulase or a mixture of cellulases. The GH61 polypeptides have recently been classified as lytic polysaccharide monooxygenases (Quinlan et al., 2011, Proc. Natl. Acad. Sci. USA 208: 15079-15084; Phillips et al., 2011, ACS Chem. Biol. 6: 1399-1406; Lin et al., 2012, Structure 20: 1051-1061) and are designated “Auxiliary Activity 9” or “AA9” polypeptides.

Hydrolytic enzymes or hydrolase/polypeptide with hydrolase activity: “Hydrolytic enzymes” refers to any catalytic protein that use water to break down substrates. Hydrolytic enzymes include cellulases (EC 3.2.1.4), xylanases (EC 3.2.1.8) arabinofuranosidases (EC 3.2.1.55 (Non-reducing end alpha-L-arabinofuranosidases); EC 3.2.1.185 (Non-reducing end beta-L-arabinofuranosidases) cellobiohydrolase I (EC 3.2.1.150), cellobiohydrolase II (E.C. 3.2.1.91), cellobiosidase (E.C. 3.2.1.176), beta-glucosidase (E.C. 3.2.1.21), beta-xylosidases (EC 3.2.1.37).

Xylanases/polypeptide with xylanase activity: The term “xylanase” means a 1,4-beta-D-xylan-xylohydrolase (E.C. 3.2.1.8) that catalyzes the endohydrolysis of 1,4-beta-D-xylosidic linkages in xylans. Xylanase activity can be determined with 0.2% AZCL-arabinoxylan as substrate in 0.01% TRITON® X-100 and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6. Xylanases can be found in, e.g., the GH5, GH8, GH30, GH10, and GH11 families.

GH5 polypeptide: refers to a polypeptide with enzyme activity, the polypeptide being classified as member of the Glycoside hydrolase family 5 in the database of Carbohydrate-Active EnZymes (CAZymes) (http://www.cazy.org/).

GH8 polypeptide: refers to a polypeptide with enzyme activity, the polypeptide being classified as member of the Glycoside hydrolase family 8 in the database of Carbohydrate-Active EnZymes (CAZymes) (http://www.cazy.org/).

GH30 polypeptide: refers to a polypeptide with enzyme activity, the polypeptide being classified as member of the Glycoside hydrolase family 30 in the database of Carbohydrate-Active EnZymes (CAZymes) (http://www.cazy.org/).

GH10 polypeptide: refers to a polypeptide with enzyme activity, the polypeptide being classified as member of the Glycoside hydrolase family 10 in the database of Carbohydrate-Active EnZymes (CAZymes) available at http://www.cazy.org/. (Lombard, V.; Golaconda Ramulu, H.; Drula, E.; Coutinho, P. M.; Henrissat, B. (21 Nov. 2013). “The carbohydrate-active enzymes database (CAZy) in 2013”. Nucleic Acids Research. 42 (D1): D490-D495; Cantarel B L, Coutinho PM, Rancurel C, Bernard T, Lombard V, Henrissat B (January 2009). “The Carbohydrate-Active EnZymes database (CAZy): an expert resource for Glycogenomics”.

Nucleic Acids Res. 37 (Database issue): D233-8).

GH11 polypeptide refers to a polypeptide with enzyme activity, the polypeptide being classified as member of the Glycoside hydrolase family 11 in the database of Carbohydrate-Active EnZymes (CAZymes).

GH62 polypeptide: refers to a polypeptide with enzyme activity, the polypeptide being classified as member of the Glycoside hydrolase family 62 in the database of Carbohydrate-Active EnZymes (CAZymes).

GH43 polypeptide: refers to a polypeptide with enzyme activity, the polypeptide being classified as member of the Glycoside hydrolase family 43 in the database of Carbohydrate-Active EnZymes (CAZymes).

GH51 polypeptide: refers to a polypeptide with enzyme activity, the polypeptide being classified as member of the Glycoside hydrolase family 51 in the database of Carbohydrate-Active EnZymes (CAZymes).

### Other Definitions:

In the present context, terms are used in manner being ordinary to a skilled person. Some of these terms are elucidated below:

Contact time: For one or more enzymes to react with a substrate, the one or more enzymes have to be in contact with the substrate. “Contact time” refers to the time period in which an effective amount of one or more enzymes is in contact with at least a fraction of a substrate mass. The enzymes may not be in contact with all of the substrate mass during the contact time, however mixing the one or more enzymes with a substrate mass allows the potential of enzymatically catalyzed hydrolysis of a fraction of the substrate mass during the contact time.

Corn kernel: A variety of corn kernels are known, including, e.g., dent corn, flint corn, pod corn, striped maize, sweet corn, waxy corn and the like.

Some corn kernels has an outer covering referred to as the “Pericarp” that protects the germ in the kernels. It resists water and water vapour and is undesirable to insects and microorganisms.

The only area of the kernels not covered by the “Pericarp” is the “Tip Cap”, which is the attachment point of the kernel to the cob.

Corn kernels or a fraction of the corn kernels: This term is used to describe the corn kernels through the process of wet milling. When the corn kernels are broken down and processed, all fractionated parts of the corn kernel are considered to be included when this term is used. The term include for example: soaked kernels, grinded kernels, corn kernel mass, a first fraction, a second fraction, one or more fractions of the corn kernel mass ect.

Corn kernel mass: is preferably used to reference a mass comprising fiber, gluten and starch, preferably achieved by steaming and grinding crop kernels and separating a mass comprising fiber, gluten and starch from germs. As the corn kernel mass move through the fiber washing, it is separated into several fractions, including a first fraction (s) and a second fraction (f). Hence, “fractions of corn kernel mass” and “one or more fractions of corn kernel mass” refer inter alia to these first (s) and second fractions (f). cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a polypeptide of the present invention. Each control sequence may be native (i.e., from the same gene) or heterologous (i.e., from a different gene) to the polynucleotide encoding the polypeptide or native or heterologous to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fragment: The term “fragment” means a polypeptide having one or more (e.g., several) amino acids absent from the amino and/or carboxyl terminus of a mature polypeptide, wherein the fragment has enzyme activity that can catalyze changes in protein disulfide bonds, including oxidation, reduction, or isomerization, e.g., a protein disulfide isomerase activity or thioredoxin peptide activity. In one embodiment a fragment comprises at least one thioredoxin domain belonging to Pfam family Pf00085.

Germ: The “Germ” is the only living part of the corn kernel. It contains the essential genetic information, enzymes, vitamins, and minerals for the kernel to grow into a corn plant. In yellow dent corn, about 25 percent of the germ is corn oil. The endosperm covered or surrounded by the germ comprises about 82 percent of the kernel dry weight and is the source of energy (starch) and protein for the germinating seed. There are two types of endosperm, soft and hard.

In the hard endosperm, starch is packed tightly together. In the soft endosperm, the starch is loose.

Gluten: Gluten is a protein, made up from two smaller proteins, glutenin and gliadin. Herein “gluten” refers to the majority of proteins found in corn kernels. In a particular embodiment, gluten refers to insoluble protein (mainly consisting of alpha-zeins, beta-zein and gamma-zeins).

The major products of gluten from corn wet milling is corn gluten meal (Approximately 60% protein) and corn gluten feed (Approximately 20% protein).

Grind or grinding: The term “grinding” refers to breaking down the corn kernels into smaller components.

Heterologous: The term “heterologous” means, with respect to a host cell, that a polypeptide or nucleic acid does not naturally occur in the host cell. The term “heterologous” means, with respect to a polypeptide or nucleic acid, that a control sequence, e.g., promoter, or domain of a polypeptide or nucleic acid is not naturally associated with the polypeptide or nucleic acid, i.e., the control sequence is from a gene other than the gene encoding the mature polypeptide of SEQ ID NO: 1, SEQ ID NO: 2, SEQ ID NO: 3, SEQ ID NO: 4, or SEQ ID NO: 5.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a polypeptide, nucleic acid, cell, or other specified material or component that is separated from at least one other material or component with which it is naturally associated as found in nature, including but not limited to, for example, other proteins, nucleic acids, cells, etc. An isolated polypeptide includes, but is not limited to, a culture broth containing the secreted polypeptide.

Incubation time: Time in which the one or more fractions of the corn kernel mass is in contact with hydrolytic enzyme during fiber washing, without being screened.

In many preferred embodiments, a method according to the present invention utilises a system comprising a space (V), or “incubator”, inside which the material is “left to be affected” by the enzymes and in such situations, the incubation time may be determined by:

$${t}_{it} = \frac{\begin{matrix}volume of incubator [ {m}^{3} ] * \\ density of inflow to incubator [ kg / {m}^{3} ]\end{matrix}}{mass inflow per time unit to the incubator [ kg / s ]}$$

Alternatively, if the inflow to the incubator is expressed in terms of volume per time unit:

$${t}_{it} = \frac{\begin{matrix}volume of incubator [ {m}^{3} ]\end{matrix}}{volume inflow per time unit to the incubator [ {m}^{3} / s ]}$$

Mature polypeptide: The term “mature polypeptide” means a polypeptide in its mature form following N-terminal processing (e.g., removal of signal peptide).

In one aspect, the mature polypeptide is amino acids 21 to 516 of SEQ ID NO: 1.

In one aspect, the mature polypeptide is amino acids 21 to 513 of SEQ ID NO: 2.

In one aspect, the mature polypeptide is amino acids 21 to 516 of SEQ ID NO: 3.

In one aspect, the mature polypeptide is amino acids 21 to 517 of SEQ ID NO: 4.

In one aspect, the mature polypeptide is amino acids 1 to 110 of SEQ ID NO: 5.

Mature polypeptide coding sequence: The term “mature polypeptide coding sequence” means a polynucleotide that encodes a mature polypeptide having protein disulfide isomerase activity or thioredoxin activity. In one aspect, the mature polypeptide coding sequence is nucleotides 61-362, 419-797, 868-1307, and 1363-1732 of SEQ ID NO: 6 In another aspect, the the mature polypeptide coding sequence is nucleotides 61-362, 427-805, 877-1316, 1372-1744 of SEQ ID NO: 7. In one aspect the mature polypeptide coding sequence is nucleotides 61-1548 of SEQ ID NO: 20. In one aspect the mature polypeptide coding sequence is nucleotides 61-1539 of SEQ ID NO: 21. In one aspect the mature polypeptide coding sequence is nucleotides 61-1548 of SEQ ID NO: 22. In one aspect the mature polypeptide coding sequence is nucleotides 61-1551 of SEQ ID NO: 23. In one aspect the mature polypeptide coding sequence is nucleotides 1-330 of SEQ ID NO: 24.

Native: The term “native” means a nucleic acid or polypeptide naturally occurring in a host cell.

Mill equipment: “Mill equipment” refers to all equipment used on a mill. The wet milling process will vary dependent on the available mill equipment. Examples of mill equipment can be steeping tanks, evaporator, screw press, rotatory dryer, dewatering screen, centrifuge, hydrocyclone ect. The size, and number of each mill equipment/milling lines can vary on different mills, which will affect the milling process. For example, the number of fiber washing screen units can vary and so can the size of a centrifuge.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Retention time: The time in which one or more hydrolytic enzymes and corn kernels or a fraction of the corn kernels are allowed to react during the fiber washing procedure.

In some embodiments, the retention time is the time period in which the corn kernel mass, received in the first screen unit (S1) and one or more fractions thereof, are contacted with an effective amount of one or more hydrolytic enzymes before leaving the fiber washing system again. During the retention time, the one or more fractions of corn kernel mass is incubated with one or more hydrolytic enzymes in a space (V), before it leaves the fiber washing system, as part of a first fraction (s1) from the most upstream screen unit (S1) or as part of a second fraction (f4) from the most downstream screen unit (S4).

Retention time may preferably be estimated as the average duration of time solid matter spends in a fiber washing system as defined in relation to the present invention. This may be estimated by the following relation:

$${t}_{rt} = \frac{volume of system : [ {m}^{3} ] * density of mass inflow [ kg / {m}^{3} ]}{mass inflow per time unit to the system [ kg / s ]}$$

Alternatively, if the inflow to the system is expressed in terms of volume per time unit:

$${t}_{it} = \frac{volume of system [ {m}^{3} ]}{volume inflow per time unit to the system [ {m}^{3} / s ]}$$

The volume of the system is typically set equal to the sum of the volumes of all voids in the system; however, as the tubing in the system typically is made small, it may be preferred to disregard the volume of the tubing.

Screened: The term “screened” or “screening” refers to the process of separating corn kernel mass into a first fraction s and a second fraction f and movement of these fractions from one screen unit to another. A non-screening period is a non-separating period provided for incubation of corn kernel mass or fractions thereof with enzymes.

Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the —nobrief option must be specified in the command line. The output of Needle labeled “longest identity” is calculated as follows:

(Identical Residues×100)/(Length of Alignment−Total Number of Gaps in Alignment)

For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:

(Identical Deoxyribonucleotides×100)/(Length of Alignment−Total Number of Gaps in Alignment)

Starch: The term “starch” means any material comprised of complex polysaccharides of plants, composed of glucose units that occurs widely in plant tissues in the form of storage granules, consisting of amylose and amylopectin, and represented as (C₆H₁₀O₅)ₙ, where n is any number.

Steeping or soaking: The term “steeping” means soaking the crop kernel with water and optionally SO₂.

### DETAILED DESCRIPTION

It is an object of the present invention to provide a method that improves starch and gluten yield from a corn wet milling process.

Particularly, it is an object of the present invention to provide a method for improving the starch and/or gluten yields that can be obtained from corn kernels in a wet milling process, by contacting ground corn kernels or a fraction of the ground kernels, particularly a fiber rich fraction, with an effective amount of a polypeptide that catalyzes changes in protein disulfide bonds, including oxidation, reduction, or isomerization. In one embodiment the polypeptide comprises at least a Pfam family domain, belonging to Pfam family Pf00085. Such polypeptides are also known as Thioredoxin polypeptides. In a particular embodiment the polypeptide comprising a Pf00085 domain is a Protein Disulfide Isomerase (PDI). The effect of the Thioredoxin polypeptide or PDI may be further improved by the presence of of at least a xylanase and/or cellulase and an effective amount of SO₂, which will further increases the release of bound starch and gluten from fiber and thus improve the starch and/or gluten yields that can be obtained.

### The Wet Milling Process:

Corn kernels are wet milled in order to open up the kernels and separate the kernels into its four main constituents: starch, germ, fiber and gluten.

The wet milling process can vary significantly from mill to mill, however conventional wet milling usually comprises the following steps:
1. Steeping
2. Grinding
3. Separation into streams comprising:
   i) germ; and ii) fiber, starch and gluten
4. Fine grinding
5. Fiber washing, pressing and drying
6. Starch/gluten separation, and
7. Starch washing.

### Steeping, Grinding and Germ Separation

Corn kernels are softened by soaking in water for between about 30 minutes to about 48 hours, preferably 30 minutes to about 15 hours, such as about 1 hour to about 6 hours at a temperature of about 50° C., such as between about 45° C. to 60° C. During steeping, the kernels absorb water, increasing their moisture levels from 15 percent to 45 percent and more than doubling in size. The optional addition of e.g. 0.1 percent sulphur dioxide (SO₂) and/or NaHSO₃ to the water prevents excessive bacteria growth in the warm environment. As the corn swells and softens, the mild acidity of the steep water begins to loosen the gluten bonds within the corn and release the starch. After the corn kernels are steeped they are cracked open to release the germ usually by a course grinding step. The germ contains corn oil. The germ is separated from the heavier density mixture of starch, gluten and fiber essentially by “floating” the germ segment free of the other substances under closely controlled conditions. This method serves to eliminate any adverse effect of traces of corn oil in later processing steps. Subsequently the germ may be dried and oil extracted. After separating the germ, the ground kernel mass comprising fiber, starch and gluten (protein) is usually subjected to a fine grinding step.

### Fiber Washing, Pressing and Drying

To get maximum starch and gluten recovery, while keeping any fiber in the final product to an absolute minimum, it is necessary to wash the free starch and gluten from the fiber during processing. Therefore the finely ground kernel mass is subjected to a fiberwashing procedure. Thereby free starch and gluten is separated from fiber during screening (washing) and collected as mill starch. The remaining fiber is then pressed to decrease the the water content.

To get maximum starch and gluten recovery, while keeping any fiber in the final product to an absolute minimum, it is necessary to wash the free starch and gluten from the fiber fraction during processing. The fiber is collected, slurried and screened, typically after soaking, grinding and separation of germs from the corn kernels, to reclaim any residual starch or gluten in the corn kernel mass. This process is herein referred to as the fiber washing procedure/step.

### Starch Gluten Separation

The starch-gluten suspension from the fiber-washing step, called mill starch, is separated into starch and gluten. Gluten has a low density compared to starch. By passing mill starch through a centrifuge, the gluten is readily spun out.

### Starch Washing

The starch slurry from the starch separation step contains some insoluble protein and much of solubles. They have to be removed before a top quality starch (high purity starch) can be made. The starch, with just one or two percent protein remaining, is diluted, washed 8 to 14 times, re-diluted and washed again in hydro-clones to remove the last trace of protein and produce high quality starch, typically more than 99.5% pure.

Products of wet milling: Wet milling can be used to produce, without limitation, corn steep liquor, corn gluten feed, germ, corn oil, corn gluten meal, corn starch, modified corn starch, syrups such as corn syrup, and corn ethanol.

An aspect of the present invention is to provide a method to increase the total starch yield and/or gluten yield that can be obtained from corn kernels in a wet milling process, the method comprising: Admixing corn kernels or a fraction of the corn kernels with an enzyme composition comprising an effective amount of a polypeptide that catalyzes changes in protein disulfide bonds, including oxidation, reduction, or isomerization. In one embodiment the polypeptide comprises at least a Pfam family domain, belonging to Pfam family Pf00085. Such polypeptides are also known as Thioredoxin polypeptides. In a particular embodiment the polypeptide comprising a Pf00085 domain is a Protein Disulfide Isomerase (PDI). The least one Protein Disulfide Isomerase or Thioredoxin polypeptide may optionally be combined with one or more hydrolytic enzymes, wherein at least one of said hydrolytic enzymes is selected from the group consisting of a xylanase polypeptide, and/or cellulase polypeptide or a combination thereof.

Other hydrolytic enzymes may also be added such as an arabinofuranosidase. In a preferred embodiment, said corn kernels or a fraction of said corn kernels is admixed with said protein disulfide isomerase or thioredoxin peptide, and optinally one or more hydrolytic enzymes during the step of subjecting the corn kernel mass to a fiber washing procedure.

Some of the starch and/or gluten in corn kernels or fractions of corn kernels, may be bound to the fiber fraction and never released during the wet milling process. However, addition of hydrolytic enzymes, which may include any catalytic protein that can use water to break down substrates present in corn kernels, may release some of the bound starch and/or gluten and thus increase the total yield of starch and/or gluten in the wet milling process.

The present inventors have surprisingly found that the release of starch and gluten from ground corn kernels can be increased by contacting the ground corn kernel mass or a fraction of the ground kernels, particularly a fiber rich fraction, with an effective amount of a Protein Disulfide Isomerase (PDI) or thioredoxin. The effect of the PDI may be further enhanced by the combined use of PDI, xylanase, cellulase, and SO2.

In a first aspect the present invention therefore relates to a method for increasing starch yield and/or gluten yield from corn kernels in a wet milling process, the method comprising contacting ground corn kernels or a fraction of the ground kernels, particularly a fiber rich fraction, with an effective amount of a polypeptide that catalyzes changes in protein disulfide bonds, including oxidation, reduction, or isomerization, such as a Protein Disulfide Isomerase (PDI) (EC 5.3.4.1) or a Thioredoxin peptide.

In one embodiment, the method of the present invention leads to an increase in the amount of starch and/or gluten released from fiber during the wet milling process compared to a process where no PDI or thioredoxin peptide is present/added.

In another embodiment, the method of the present invention leads to a reduction in steeping time and SO₂ addition.

The specific procedure and the equipment used in the wet milling process can vary, but the main principles of the process remains the same (See description on wet milling process).

In one embodiment the PDI or thioredoxin peptide is present/added to a fiber fraction, particularly during a fiber washing step.

In one specific embodiment, the method of the invention comprise the steps of:
a) soaking the corn kernels in water to produce soaked kernels;
b) grinding the soaked kernels to produce ground kernels;
c) separating germ from the ground kernels to produce a corn kernel mass comprising fiber, starch and gluten; and
d) subjecting the corn kernel mass, particularly fiber rich fraction, such as a fine fiber fraction, to a fiber washing procedure separating starch and gluten from the fiber; wherein at least a PDI or thioredoxin peptide is present/added before or during step d).

In one embodiment the above method further comprising the steps of:
e) separating the starch from the gluten; and optionally
f) washing the starch.

In one embodiment of the method of the invention the PDI or Thioredoxin peptide is present/added during fiber wash in amounts at least 10 μg EP/g DS, at least 25 μg EP/g DS, at least 50 μg EP/g DS, at least 75 μg EP/g DS, at least 100 μg EP/g DS, at least 200 μg EP/g DS, at least 300 μg EP/g DS, at least 500 μg EP/g DS, such as in the range from 10 to 2000 μg EP/g DS, 25 to 1000 μg EP/g DS, 50 to 800 μg EP/g DS, 100 to 500 μg EP/g DS.

According to the invention, in order to maximize the effect of the hydrolytic enzymes during the fiber washing step, an effective amount of SO₂ is present during the fiber wash.

In one embodiment SO₂ is present/added during fiber wash in amounts of at least 200 ppm, at least 300 ppm, at least 400 ppm, at least 450 ppm, at least 500 ppm, at least 600 ppm, at least 700 ppm, at least 800 ppm.

In another embodiment SO2 is present/added during fiber wash in amounts in a range from 200-3000 ppm, 300-2000 ppm, 400-800 ppm.

In the method according to the invention, further enzyme activities may advantageously be added in combination with the PDI or thioredoxin peptide. Therefore a further embodiment of the invention relates to a method, wherein an effective amount of one or more hydrolytic enzymes are present/added before or during the fiber washing step, and wherein at least one of said hydrolytic enzymes is selected from xylanases and/or cellulases.

The xylanase are in one embodiment selected from the group consisting of a GH5 polypeptide, GH30 polypeptide, a GH10 polypeptide, a GH11 polypeptide, a GH8 polypeptide or a combination thereof.

In another embodiment, the hydrolytic enzymes comprise one or more cellulases. The cellulase(s) comprises one or more enzyme selected from the group consisting of an endoglucanase (EG), and a cellobiohydrolase (CBH).

In one embodiment the cellulase(s) comprises one or more enzyme selected from the group consisting of an endoglucanase, a cellobiohydrolase I, a cellobiohydrolase II, or a combination thereof.

In another embodiment the hydrolytic enzymes comprise an arabinofuranosidase.

The arabinofurasnosidase may in one embodiment be selected from the group consisting of a GH43 polypeptide, a GH62 polypeptide, and a GH51 polypeptide.

In one particular embodiment of the method of the invention, the corn kernel mass comprising fiber, starch and gluten from step c) is subjected to a further grinding step, preferably a fine grinding step before the fiber washing procedure in step d).

The specific equipment used in the fiber washing procedure may vary, but the main principle of the process remains the same. WO2018/053220 describes a fiber-washing system including a dedicated enzyme incubation space/tank. Based on this disclosure and the general knowledge of the skilled person it will be possible to design a fiber-washing system resulting in sufficient incubation time for the hydrolytic enzymes to work. In one embodiment, said corn kernels or a fraction of said corn kernels, e.g., a fiber rich fraction, is allowed to react with said one or more hydrolytic enzymes for at least 15 minutes, such as at least 20 minutes, at least 25 minutes, at least 30 minutes, at least 35 minutes, at least 40 minutes, at least 45 minutes, at least 50 minutes, at least 55 minutes, at least 60 minutes, at least 70 minutes, at least 80 minutes, at least 90 minutes, at least 100 minutes, at least 110 minutes or at least 120 minutes.

In one embodiment, said fiber washing procedure comprise the use of a fiber washing system optimized for introduction of one or more hydrolytic enzymes, wherein the fiber washing system comprise a space (V) configured to provide a total reaction time in the fiber washing system (retention time) of at least 35 minutes, such as at least 40 minutes, at least 45 minutes, at least 50 minutes, at least 60 minutes, at least 70 minutes, at least 80 minutes, at least 90 minutes, at least 100 minutes, at least 110 minutes or at least 120 minutes and less than 48 hours, such as less than 40 hours, less than 36 hours, less than 30 hours, less than 24 hours, less than 20 hours, less than 12 hours, less than 10 hours, less than 8 hours, less than 6 hours, less than 5 hours, less than 4 hours, less than 3 hours. In one embodiment the total retention time in the fiber washing system is between 35 minutes and 48 hours such as between 35 minutes and 24 hours, 35 minutes and 12 hours, 35 minutes and 6 hours, 35 minutes and 5 hours, 35 minutes and 4 hours, 35 minutes and 3 hours, 35 minutes and 2 hours, 45 minutes and 48 hours, 45 minutes and 24 hours, 45 minutes and hours, 45 minutes and 6 hours, 45 minutes and 5 hours, 45 minutes and 4 hours, 45 minutes and 3 hours, 45 minutes and 2 hours 1-48 hours, 1-24 hours, 1-12 hours, 1-6 hours, 1-5 hours, 1-4 hours, 1-3 hours, 1-2 hours.

In one embodiment, the fiber washing system comprises:
a plurality of screen units (S1 . . . S4) being fluidly connected in a counter current washing configuration; each screen unit being configured for separating a stream of corn kernel mass and liquid into two fractions: a first fraction (s) and a second fraction (f), said second fraction (f) containing a higher amount measured in wt % fiber than the first fraction (s);
a space (V) arranged in the system and being fluidly connected to receive said first fraction (s), said second fraction (f), or a mixed first and second fraction (s,f), preferably only a second fraction (f), and configured to provide an incubation time for one or both fractions received in the space; and outletting the thereby incubated one or both fractions to a downstream screen unit (S4),

wherein the system is configured for
inletting corn kernel mass and liquid to the most upstream screen unit (S1)
outletting the first fraction (s1) from the most upstream screen unit (S1) as a product stream containing starch,
inletting process water, preferably arranged for inletting process water to a most downstream screen unit (S4),
outletting the second fraction (f4) from most downstream screen unit (S4) as a washed corn kernel mass containing a lower amount of starch and gluten than the original corn kernel mass.
introducing hydrolytic enzymes into the system.

In one embodiment, the incubation time in said space (V) configured into the fiber washing system is at least 5 minutes such as at least 10 minutes, at least 15 minutes, at least 20 minutes, at least 30 minutes, at least 40 minutes, at least 45 minutes, at least 50 minutes, at least 60 minutes, at least 70 minutes, at least 80 minutes, at least 90 minutes, at least 100 minutes, at least 110 minutes or at least 120 minutes and less than 48 hours, such as less than 40 hours, less than 36 hours, less than 30 hours, less than 24 hours, less than 20 hours, less than 12 hours, less than 10 hours, less than 8 hours, less than 6 hours, less than 5 hours, less than 4 hours, less than 3 hours.

In one embodiment the incubation time in said space (V) is between 35 minutes and 48 hours such as between 35 minutes and 24 hours, 35 minutes and hours, 35 minutes and 6 hours, 35 minutes and 5 hours, 35 minutes and 4 hours, 35 minutes and 3 hours, 35 minutes and 2 hours, 45 minutes and 48 hours, 45 minutes and 24 hours, 45 minutes and 12 hours, 45 minutes and 6 hours, 45 minutes and 5 hours, 45 minutes and 4 hours, 45 minutes and 3 hours, 45 minutes and 2 hours 1-48 hours, 1-24 hours, 1-12 hours, 1-6 hours, 1-5 hours, 1-4 hours, 1-3 hours, 1-2 hours.

In one embodiment, the incubation temperature in said space (V) is between 25 and 95° C., such as between 25 and 90° C., 25 and 85° C., 25 and 80° C., 25 and 75° C., 25 and 70° C., 25 and 65° C., 25 and 60° C., 25 and 55° C., 25 and 53° C., 25 and 52° C., 30 and 90° C., 30 and 85° C., 30 and 80° C., 30 and 75° C., 30 and 70° C., 30 and 65° C., 30 and 60° C., 30 and 55° C., 30 and 53° C., 30 and 52° C., 35 and 90° C., 35 and 85° C., 35 and 80° C., 35 and 75° C., 35 and 70° C., 35 and 65° C., 35 and 60° C., 35 and 55° C., 35 and 53° C., 35 and 52° C., 39 and 90° C., 39 and 85° C., 39 and 80° C., 39 and 75° C., 39 and 70° C., 39 and 65° C., 39 and 60° C., 39 and 55° C., 39 and 53° C., 39 and 52° C., such as 46 and 52° C.

Further, the dimension of the space (in m³) is preferably configured to provide an incubation time of at least at least 5 minutes, such as at least 10 minutes, at least 15 minutes, at least 20 minutes at least 25 minutes, at least 30 minutes, at least 35 minutes, at least 40 minutes, at least 45 minutes, at least 50 minutes, at least 55 minutes, at least 60 minutes, at least 70 minutes, at least 80 minutes, at least 90 minutes, at least 100 minutes, at least 110 minutes, at least 120 minutes.

The space (V) designated for incubation preferably has a volume of at least 30 m³, at least 40 m³, at least 50 m³, at least 60 m³, at least 70, m³, at least 80, m³, at least 90, m³, at least 100 m³, at least 110 m³, at least 120 m³, at least 130 m³, at least 140 m³, at least 150 m³, at least 160 m³, at least 170 m³, at least 180 m³, at least 190 m³, at least 200 m³, at least 210 m³, at least 220 m³, at least 230 m³, at least 240 m³, at least 250 m³, at least 260 m³, at least 270 m³, at least 280 m³, at least 290 m³, at least 300 m³, at least 400 m³, or at least 500 m³. The incubation time may also be in more than one space V with a total or combined volume of at least 100 m³, at least 110 m³, at least 120 m³, at least 130 m³, at least 140 m³, at least 150 m³, at least 160 m³, at least 170 m³, at least 180 m³, at least 190 m³, at least 200 m³, at least 210 m³, at least 220 m³, at least 230 m³, at least 240 m³, at least 250 m³, at least 260 m³, at least 270 m³, at least 280 m³, at least 290 m³, at least 300 m³, at least 400 m³, at least 500 m³.

During the incubation time, it is preferred that the fluid received in the space V is not screened. Thus, the fluid leaving the space V has the same composition, e.g. of starch and fiber, as the fluid received in the space V, although it preferably contains a higher proportion of starch and/or protein that has been released from the fibers.

To assure intimate contact between the enzymes and the fiber, it may be preferred to configure the space V for agitation of matter contained in said space V, such as by comprising a rotor or impeller.

It is preferred to arrange the space V downstream of the most upstream screen unit S1 and upstream of said most downstream screen unit S4; in particular, the space V is arranged to feed fluid into the second most downstream screen unit S3.

### Protein Disulfide Isomerase, EC 5.3.4.1.

In one embodiment the PDI comprises at least one catalytic domain belonging to the thioredoxin superfamily of redox proteins, and wherein this domain comprises an active site characterized by having a CXXC motif.

In another embodiment the PDI comprises two catalytic domains, each comprising an active site characterized by having a CXXC motif, separated by at least one non-catalytic domains.

The CXXC motif is in particular selected from the group consisting of CGHC, CTHC, CPHC, and CSMC, most particularly CGHC.

In a particular embodiment the thioredoxin domains belong to either Pfam family Pf00085 or Pf13848.

In another particular embodiment the PDI comprises a three domain structure Pf00085-Pf13848-Pf00085 in which the catalytically active domains are denoted Pf00085.

### Thioredoxin Peptide,

In one embodiment thioredoxins are proteins that act as antioxidants by facilitating the reduction of other proteins by cysteine thiol-disulfide exchange. They are enzymes that can catalyze the reduction of protein disulfide bonds. In another embodiment the thioredoxin peptide comprises an active site motif CXXC, particularly CGPC. Thioredoxin proteins also have a characteristic tertiary structure termed the thioredoxin fold. Therefore, according to this invention a thioredoxin peptide is a member of the thioredoxin superfamily of redox proteins, and typically comprise a catalytic domain comprising an active site characterized by having a CXXC, preferably a CGPC motif. The thioredoxin domain belongs to Pfam family Pf00085 (https://pfam.xfam.org/family/Thioredoxin).

### Hydrolytic Enzymes Suitable for the Method of the Invention in Addition to PDI or Trx

In one embodiment, hydrolytic enzymes suitable for use in the method of the invention comprise one or more enzymes selected form the group consisting of: cellulases (EC 3.2.1.4), xylanases (EC 3.2.1.8), arabinofuranosidases (EC 3.2.1.55 (Non-reducing end alpha-L-arabinofuranosidases); EC 3.2.1.185 (Non-reducing end beta-L-arabinofuranosidases), cellobiohydrolase I (EC 3.2.1.150), cellobiohydrolase II (E.C. 3.2.1.91), cellobiosidase (E.C. 3.2.1.176), beta-glucosidase (E.C. 3.2.1.21), beta-xylosidases (EC 3.2.1.37), and proteases (E.C 3.4).

Preferably the the enzymes are selected from xylanase and/or cellulases.

In one embodiment the xylanase is selected from the group consisting of a GH5 polypeptide, GH30 polypeptide, a GH10 polypeptide, a GH11 polypeptide, a GH8 polypeptide or a combination thereof.

In another embodiment the hydrolytic enzymes comprise one or more cellulases. The cellulases may be selected from at least the group consisting of an endoglucanase (EG), and a cellobiohydrolase (CBH). More particularly, the cellulase(s) comprises one or more enzyme selected from the group consisting of an endoglucanase, a cellobiohydrolase I, a cellobiohydrolase II, or a combination thereof.

In one embodiment the hydrolytic enzymes further comprise an arabinofuranosidase. The arabinofuranosidase may be selected from the group consisting of a GH43 polypeptide, a GH62 polypeptide, GH51 polypeptide. Particularly a GH62 polypeptide.

In one embodiment, the one or more hydrolytic enzymes is expressed in an organism with a cellulase background, such as Trichoderma reesei. According to these embodiments the xylanase and or arabinofuranosidase polypeptides defined according to the invention is/are expressed together with endogenous cellulases from Trichoderma.

In one embodiment, the enzyme composition comprising one or more hydrolytic enzymes may comprise cellulases derived from Trichoderma reesei or Humicula insolens.

In one particular embodiment the celullases are derived from Trichoderma reesei background cellulases and having a CBH I and CBH II derived from Aspergillus fumigatus.

In one embodiment, the cellulase enzyme composition comprises Aspergillus fumigatus GH10 xylanase (WO 2006/078256) and Aspergillus fumigatus beta-xylosidase (WO 2011/057140) with a Trichoderma reesei cellulase preparation containing Aspergillus fumigatus cellobiohydrolase I (WO 2011/057140), Aspergillus fumigatus cellobiohydrolase II (WO 2011/057140), Aspergillus fumigatus beta-glucosidase variant (WO 2012/044915), and Penicillium sp. (emersonii) GH61 polypeptide (WO 2011/041397), wherein endoglucanase activity is provided from the Trichoderma reesei cellulases.

In another particular embodiment the cellulase enzyme composition comprises a Trichoderma reesei cellulase preparation containing Aspergillus fumigatus cellobiohydrolase I (WO 2011/057140), Aspergillus fumigatus cellobiohydrolase II (WO 2011/057140), Aspergillus fumigatus beta-glucosidase variant (WO 2012/044915), and Penicillium sp. (emersonii) GH61 polypeptide (WO 2011/041397), wherein endoglucanase activity is provided from the Trichoderma reesei cellulases.

In one embodiment, the one or more hydrolytic enzymes are purified. The purified enzymes may be used in an enzyme composition as described in other embodiments of the present invention.

In one embodiment, the one or more hydrolytic enzymes is/are in a liquid composition. The composition may be homogenous or heterogeneous.

In one embodiment, the one or more hydrolytic enzymes is/are in a solid composition.

In one embodiment, the effective amount of one or more hydrolytic enzymes admixed with one or more fractions of said corn kernel mass, is between 0.005-0.5 kg enzyme protein (EP)/metric tonne (MT) corn kernels entering the wet milling process, such as between 0.010-0.5 kg EP/MT corn kernel, such as between 0.05-0.5 kg/MT corn kernel or 0.075-0.5 kg/MT or 0.1-0.5 kg/MT corn kernel or 0.005-0.4 kg/MT corn kernel or 0.01-0.4 kg/MT corn kernel or 0.05-0.4 kg/MT corn kernel or 0.075-0.4 kg/MT corn kernel or 0.1-0.4 kg/MT corn kernel or 0.005-0.3 kg/MT corn kernel or 0.01-0.3 kg/MT corn kernel or 0.05-0.3 kg/MT corn kernel or 0.075-0.3 kg/MT or 0.1-0.3 kg/MT corn kernel or 0.005-0.2 kg/MT corn kernel or 0.010-0.2 kg/MT corn kernel or 0.05-0.2 kg/MT corn kernel or 0.075-0.2 kg/MT or 0.1-0.2 kg/MT corn kernel or such as 0.075-0.10 kg/MT corn kernel or 0.075-0.11 kg/MT corn kernel.

In preferred embodiments the enzyme composition comprises cellulase obtained from a culture of Trichoderma reesei, such as a culture of Trichoderma reesei ATCC 26921. Suitable cellulases are available; e.g. from Novozymes A/S under the commercial name Celluclast®.

### Polypeptides Having Xylanase Activity

Xylanases are suitable to be applied in the method according to the invention. The xylanase polypeptide may be selected from family GH5, GH10, GH30, GH11, and GH8.

More specific embodiments relates to the method according to the invention, wherein the GH5 xylanase enzyme is selected from the group consisting of:
(a) a polypeptide having at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 8;
(b) a variant of the mature polypeptide of SEQ ID NO: 8 comprising a substitution, deletion, and/or insertion at one or more positions; and
(c) a fragment of the polypeptide of (a), or (b) that has xylanase activity.

The mature polypeptide is in one embodiment amino acids 1 to 551 of SEQ ID NO: 8.

Another specific embodiments relates to the method according to the invention, wherein the GH5 xylanase enzyme is selected from the group consisting of:
(a) a polypeptide having at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 9;
(b) a variant of the mature polypeptide of SEQ ID NO: 9 comprising a substitution, deletion, and/or insertion at one or more positions; and
(c) a fragment of the polypeptide of (a), or (b) that has xylanase activity.

The mature polypeptide is in one embodiment amino acids 1 to 551 of SEQ ID NO: 9.

Another specific embodiment relates to the method according to the invention, wherein the GH10 xylanase is selected from the group consisting of:
(a) a polypeptide having at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 11;
(b) a variant of the mature polypeptide of SEQ ID NO: 11 comprising a substitution, deletion, and/or insertion at one or more positions; and
(c) a fragment of the polypeptide of (a), or (b) that has xylanase activity.

The mature polypeptide is in one embodiment amino acids 21 to 405 of SEQ ID NO: 11.

Another specific embodiment relates to the method according to the invention, wherein the GH10 xylanase is selected from the group consisting of:
(a) a polypeptide having at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 10;
(b) a variant of the mature polypeptide of SEQ ID NO: 10 comprising a substitution, deletion, and/or insertion at one or more positions; and
(c) a fragment of the polypeptide of (a), or (b) that has xylanase activity.

The mature polypeptide is in one embodiment amino acids 20 to 319 of SEQ ID NO: 10.

Polypeptides Having Arabinofuranosidase Activity Another specific embodiment relates to the method according to the invention, wherein the the GH62 arabinofuranosidase is selected from the group consisting of:
(a) a polypeptide having at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 12;
(b) a variant of the mature polypeptide of SEQ ID NO: 12 comprising a substitution, deletion, and/or insertion at one or more positions; and
(c) a fragment of the polypeptide of (a), or (b) that has arabinofuranosidase activity.

The mature polypeptide is in one embodiment amino acids 27 to 332 of SEQ ID NO: 12.

Another specific embodiment relates to the method according to the invention, wherein the the GH62 arabinofuranosidase is selected from the group consisting of:
(a) a polypeptide having at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 13;
(b) a variant of the mature polypeptide of SEQ ID NO: 13 comprising a substitution, deletion, and/or insertion at one or more positions; and
(c) a fragment of the polypeptide of (a), or (b) that has arabinofuranosidase activity.

The mature polypeptide is in one embodiment amino acids 17 to 325 of SEQ ID NO: 13.

Polypeptides Having Protein Disulfide Isomerase (PDI) Activity or Thioredoxin Activity In some embodiments, the present invention relates to isolated or purified polypeptides having a sequence identity of at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 1, which have protein disulfide isomerase activity, and preferably the PDI activity is at least 75% of the PDI activity of the mature polypeptide of SEQ ID NO: 1, at least 80%, at least 85%, at least 90%, at least 95%, or at least 99% of the activity of the mature polypeptide of SEQ ID NO: 1. In one aspect, the polypeptides differ by up to 10 amino acids, e.g., 1, 2, 3, 4, 5, 6, 7, 8, 9, or 10, from the mature polypeptide of SEQ ID NO: 1.

The polypeptide preferably comprises, consists essentially of, or consists of the amino acid sequence of SEQ ID NO: 1 or the mature polypeptide thereof; or is a fragment thereof having PDI activity. In one aspect, the mature polypeptide is amino acids 21 to 516 of SEQ ID NO: 1.

In one embodiment the polypeptide having PDI activity is encoded by a polynucleotide having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide encodimg sequence of SEQ ID NO: 20.

In some embodiments, the present invention relates to isolated or purified polypeptides having a sequence identity of at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 2, which have protein disulfide isomerase activity, and preferably the PDI activity is at least 75% of the PDI activity of the mature polypeptide of SEQ ID NO: 2, at least 80%, at least 85%, at least 90%, at least 95%, or at least 99% of the activity of the mature polypeptide of SEQ ID NO: 2. In one aspect, the polypeptides differ by up to 10 amino acids, e.g., 1, 2, 3, 4, 5, 6, 7, 8, 9, or 10, from the mature polypeptide of SEQ ID NO: 2. The polypeptide preferably comprises, consists essentially of, or consists of the amino acid sequence of SEQ ID NO: 2 or the mature polypeptide thereof; or is a fragment thereof having PDI activity. In one aspect, the mature polypeptide is amino acids 21 to 513 of SEQ ID NO: 2.

In one embodiment the polypeptide having PDI activity is encoded by a polynucleotide having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide encodimg sequence of SEQ ID NO: 21.

In some embodiments, the present invention relates to isolated or purified polypeptides having a sequence identity of at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 3, which have protein disulfide isomerase activity, and preferably the PDI activity is at least 75% of the PDI activity of the mature polypeptide of SEQ ID NO: 3, at least 80%, at least 85%, at least 90%, at least 95%, or at least 99% of the activity of the mature polypeptide of SEQ ID NO: 3. In one aspect, the polypeptides differ by up to 10 amino acids, e.g., 1, 2, 3, 4, 5, 6, 7, 8, 9, or 10, from the mature polypeptide of SEQ ID NO: 3.

The polypeptide preferably comprises, consists essentially of, or consists of the amino acid sequence of SEQ ID NO: 3 or the mature polypeptide thereof; or is a fragment thereof having PDI activity. In one aspect, the mature polypeptide is amino acids 21 to 516 of SEQ ID NO: 3.

In one embodiment the polypeptide having PDI activity is encoded by a polynucleotide having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide encodimg sequence of SEQ ID NO: 22.

In some embodiments, the present invention relates to isolated or purified polypeptides having a sequence identity of at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 4, which have protein disulfide isomerase activity, and preferably the PDI activity is at least 75% of the PDI activity of the mature polypeptide of SEQ ID NO: 4, at least 80%, at least 85%, at least 90%, at least 95%, or at least 99% of the activity of the mature polypeptide of SEQ ID NO: 4. In one aspect, the polypeptides differ by up to 10 amino acids, e.g., 1, 2, 3, 4, 5, 6, 7, 8, 9, or 10, from the mature polypeptide of SEQ ID NO: 4.

The polypeptide preferably comprises, consists essentially of, or consists of the amino acid sequence of SEQ ID NO: 4 or the mature polypeptide thereof; or is a fragment thereof having PDI activity. In one aspect, the mature polypeptide is amino acids 21 to 517 of SEQ ID NO: 4.

In one embodiment the polypeptide having PDI activity is encoded by a polynucleotide having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide encodimg sequence of SEQ ID NO: 23.

In some embodiments, the present invention relates to isolated or purified thioredoxin polypeptides having a sequence identity of at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 5, which have thioredoxin peptide activity, and preferably the thiredoxin activity is at least 75% of the thiredoxin activity of the mature polypeptide of SEQ ID NO: 5, at least 80%, at least 85%, at least 90%, at least 95%, or at least 99% of the activity of the mature polypeptide of SEQ ID NO: 5. In one aspect, the polypeptides differ by up to 10 amino acids, e.g., 1, 2, 3, 4, 5, 6, 7, 8, 9, or 10, from the mature polypeptide of SEQ ID NO: 5.

The polypeptide preferably comprises, consists essentially of, or consists of the amino acid sequence of SEQ ID NO: 5 or the mature polypeptide thereof; or is a fragment thereof having thioredoxin peptide activity. In one aspect, the mature polypeptide is amino acids 1 to 110 of SEQ ID NO: 5.

In one embodiment the polypeptide having thioredoxin activity is encoded by a polynucleotide having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide encodimg sequence of SEQ ID NO: 24.

Sources of Polypeptides Having Protein Disulfide Isomerase Activity or Thioredoxin peptide Activity

A polypeptide having Protein Disulfide Isomerase activity of the present invention may be obtained from any suitable source. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

In another aspect, the polypeptide is a polypeptide obtained from a strain of Themoascus, particularly a Thermoascus crustaceus, e.g., a polypeptide obtained from Themoascus crustaceus CBS181.67.

In another aspect, the polypeptide is a polypeptide obtained from a strain of Keithomyces, particularly Keithomyces carneus.

In another aspect, the polypeptide is a polypeptide obtained from a strain of Aspergillus, particularly Aspergillus spinulosporus.

In another aspect, the polypeptide is a polypeptide obtained from a strain of Thermoascus, particularly Thermoascus aurantiacus.

In another aspect, the polypeptide is a polypeptide obtained from Aspergillus aculeatus, e.g., a polypeptide obtained from Aspergillus aculeatus CBS101.43.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

The polypeptides may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample.

Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Polynucleotides

The present invention also relates to isolated polynucleotides encoding a polypeptide of the present invention, as described herein.

In one embodiment, the present invention relates to isolated or purified polynucleotides having a sequence identity of at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 20.

In one embodiment, the present invention relates to isolated or purified polynucleotides having a sequence identity of at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 21.

In one embodiment, the present invention relates to isolated or purified polynucleotides having a sequence identity of at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 22.

In one embodiment, the present invention relates to isolated or purified polynucleotides having a sequence identity of at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 23.

In one embodiment, the present invention relates to isolated or purified polynucleotides having a sequence identity of at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 24.

The techniques used to isolate or clone a polynucleotide are known in the art and include isolation from genomic DNA or cDNA, or a combination thereof. The cloning of the polynucleotides from genomic DNA can be effected, e.g., by using the polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Thermoascus, Keithomyces, or Aspergillus, or a related organism and thus, for example, may be a species variant of the polypeptide encoding region of the polynucleotide.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as the mature polypeptide coding sequence of SEQ ID NO: 6 or SEQ ID NO: 7 or the cDNA sequences thereof, e.g., a subsequence thereof, and/or by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

### Nucleic Acid Constructs

The present invention also relates to nucleic acid constructs comprising a polynucleotide of the present invention, wherein the polynucleotide is operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell. In one embodiment the control sequence(s) are native or heterologous to the polynucleotides encoding the polypeptides of the invention.

Examples of suitable promoters for directing transcription of the polynucleotide of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryll/A gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the polynucleotide of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase Ill, Trichoderma reesei endoglucanase V, Trichoderma reesei xylanase I, Trichoderma reesei xylanase II, Trichoderma reesei xylanase Ill, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase Ill, Trichoderma reesei endoglucanase V, Trichoderma reesei xylanase I, Trichoderma reesei xylanase II, Trichoderma reesei xylanase Ill, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cry/I/A gene (WO 94/25612) and a Bacillus subtilis SP82 gene (Hue et al., 1995, J. Bacteriol. 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is heterologous to the coding sequence. A heterologous signal peptide coding sequence may be required where the coding sequence does not naturally contain a signal peptide coding sequence. Alternatively, a heterologous signal peptide coding sequence may simply replace the natural signal peptide coding sequence to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiol. Rev. 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites.

Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome.

The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3.

Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is a hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMB1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier.

The choice of a host cell will to a large extent depend upon the gene encoding the polypeptide and its source.

In some embodiments, the polypeptide is heterologous to the recombinant host cell.

In some embodiments, at least one of the one or more control sequences is heterologous to the polynucleotide encoding the polypeptide.

In some embodiments, the recombinant host cell comprises at least two copies, e.g., three, four, or five, of the polynucleotide of the present invention.

The host cell may be any microbial or plant cell useful in the recombinant production of a polypeptide of the present invention, e.g., a prokaryotic cell or a fungal cell.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se.

Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a polypeptide of the present invention, comprising (a) cultivating a recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid-state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide The polypeptide may be recovered using methods known in the art. For example, the polypeptide may be recovered from the fermentation medium by conventional procedures including, but not limited to, collection, centrifugation, filtration, extraction, spray-drying, evaporation, or precipitation. In one aspect, a whole fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

### Enzyme Compositions

An enzyme composition for use in the method according to the invention may comprise an enzyme that catalyzes changes in protein disulfide bonds, including oxidation, reduction, or isomerization, such as a Protein Disulfide Isomerase (PDI) (EC 5.3.4.1) or a Thioredoxin peptide as the major enzymatic component, e.g., a mono-component composition. Alternatively, the compositions may comprise multiple enzymatic activities, such as one or more (e.g., several) enzymes selected from the group consisting of xylanse, cellobiohydrolase, cellulase, endoglucanase, and/or arabinofuranosidase.

The compositions may be prepared in accordance with methods known in the art and may be in the form of a liquid or a dry composition. The compositions may be stabilized in accordance with methods known in the art.

### Liquid Formulations

The present invention also relates to liquid compositions comprising the protein disulfide isomerases or thioredoxin peptides of the invention. The composition may comprise an enzyme stabilizer (examples of which include polyols such as propylene glycol or glycerol, sugar or sugar alcohol, lactic acid, reversible protease inhibitor, boric acid, or a boric acid derivative, e.g., an aromatic borate ester, or a phenyl boronic acid derivative such as 4-formylphenyl boronic acid).

In some embodiments, filler(s) or carrier material(s) are included to increase the volume of such compositions. Suitable filler or carrier materials include, but are not limited to, various salts of sulfate, carbonate and silicate as well as talc, clay and the like. Suitable filler or carrier materials for liquid compositions include, but are not limited to water or low molecular weight primary and secondary alcohols including polyols and diols. Examples of such alcohols include, but are not limited to, methanol, ethanol, propanol and isopropanol. In some embodiments, the compositions contain from about 5% to about 90% of such materials.

In an aspect, the present invention relates to liquid formulations comprising:
(A) 0.001% to 25% w/w of one or more polypeptides having [enzyme] activity of the present invention; and
(B) water.

In another embodiment, the liquid formulation comprises 20% to 80% w/w of polyol. In one embodiment, the liquid formulation comprises 0.001% to 2.0% w/w preservative.

In another embodiment, the invention relates to liquid formulations comprising:
(A) 0.001% to 25% w/w of one or more polypeptides having [enzyme] activity of the present invention;
(B) 20% to 80% w/w of polyol;
(C) optionally 0.001% to 2.0% w/w preservative; and
(D) water.

In another embodiment, the invention relates to liquid formulations comprising:
(A) 0.001% to 25% w/w of one or more polypeptides having [enzyme] activity of the present invention;
(B) 0.001% to 2.0% w/w preservative;
(C) optionally 20% to 80% w/w of polyol; and
(D) water.

In another embodiment, the liquid formulation comprises one or more formulating agents, such as a formulating agent selected from the group consisting of polyol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, PVA, acetate and phosphate, preferably selected from the group consisting of sodium sulfate, dextrin, cellulose, sodium thiosulfate, kaolin and calcium carbonate. In one embodiment, the polyols is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600, more preferably selected from the group consisting of glycerol, sorbitol and propylene glycol (MPG) or any combination thereof.

In another embodiment, the liquid formulation comprises 20%-80% polyol (i.e., total amount of polyol), e.g., 25%-75% polyol, 30%-70% polyol, 35%-65% polyol, or 40%-60% polyol.

In one embodiment, the liquid formulation comprises 20%-80% polyol, e.g., 25%-75% polyol, 30%-70% polyol, 35%-65% polyol, or 40%-60% polyol, wherein the polyol is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600. In one embodiment, the liquid formulation comprises 20%-80% polyol (i.e., total amount of polyol), e.g., 25%-75% polyol, 30%-70% polyol, 35%-65% polyol, or 40%-60% polyol, wherein the polyol is selected from the group consisting of glycerol, sorbitol and propylene glycol (MPG).

In another embodiment, the preservative is selected from the group consisting of sodium sorbate, potassium sorbate, sodium benzoate and potassium benzoate or any combination thereof. In one embodiment, the liquid formulation comprises 0.02% to 1.5% w/w preservative, e.g., 0.05% to 1.0% w/w preservative or 0.1% to 0.5% w/w preservative. In one embodiment, the liquid formulation comprises 0.001% to 2.0% w/w preservative (i.e., total amount of preservative), e.g., 0.02% to 1.5% w/w preservative, 0.05% to 1.0% w/w preservative, or 0.1% to 0.5% w/w preservative, wherein the preservative is selected from the group consisting of sodium sorbate, potassium sorbate, sodium benzoate and potassium benzoate or any combination thereof.

In another embodiment, the liquid formulation further comprises one or more additional enzymes, e.g., hydrolase, isomerase, ligase, lyase, oxidoreductase, and transferase. The one or more additional enzymes are preferably selected from the group consisting of acetylxylan esterase, acylglycerol lipase, amylase, alpha-amylase, beta-amylase, arabinofuranosidase, cellobiohydrolases, cellulase, feruloyl esterase, galactanase, alpha-galactosidase, beta-galactosidase, beta-glucanase, beta-glucosidase, lysophospholipase, lysozyme, alpha-mannosidase, beta-mannosidase (mannanase), phytase, phospholipase A1, phospholipase A2, phospholipase D, protease, pullulanase, pectin esterase, triacylglycerol lipase, xylanase, beta-xylosidase or any combination thereof.

### Fermentation Broth Formulations or Cell Compositions

The present invention also relates to a fermentation broth formulation or a cell composition comprising a polypeptide of the present invention. The fermentation broth formulation or the cell composition further comprises additional ingredients used in the fermentation process, such as, for example, cells (including, the host cells containing the gene encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation.

Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In some embodiments, the fermentation broth formulation or the cell composition comprises a first organic acid component comprising at least one 1-5 carbon organic acid and/or a salt thereof and a second organic acid component comprising at least one 6 or more carbon organic acid and/or a salt thereof. In some embodiments, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In some embodiments, the killed cells and/or cell debris are removed from a cell-killed whole broth to provide a composition that is free of these components.

The fermentation broth formulation or cell composition may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell composition of the present invention may be produced by a method described in WO 90/15861 or WO 2010/096673.

The present invention is further disclosed in the following numbered embodiments.

Embodiment [1]. A method for increasing starch yield and/or gluten yield from corn kernels in a wet milling process, the method comprising contacting ground corn kernels, particularly a fiber rich fraction of the ground kernels, with an effective amount of a polypeptide that catalyzes changes in protein disulfide bonds, including oxidation, reduction, or isomerization, such as a Protein Disulfide Isomerase (PDI) (EC 5.3.4.1) or a Thioredoxin peptide.

Embodiment [2]. The method according to embodiment 1, wherein the amount of starch and/or gluten, particularly insoluble starch and gluten, released from ground kernels during the wet milling process is increased compared to a process where no PDI or Thioredoxin peptide is present/added.

Embodiment [3]. The method according to embodiment 2, wherein the increase measured as mg protein released per gram dry solids (mg/gDS) is at least 0.5% points, at least 0.75% points, at least 1.0% points, at least 1.5% points, at least 2.5% points, such as at least 5.0% points, compared to no addition of PDI, wherein fiber-wash is performed at a pH in the range from 3.5-5.5, such as at pH 4.0, 4.5 or 5.0, and an enzyme dosage of at least 300 μg EP/gDS.

Embodiment [4]. The method of any of embodiments 1-3, wherein the PDI or Thioredoxin peptide is present/added to a fiber fraction, particularly during a fiber washing step.

Embodiment [5]. The method according to any of the preceding embodiments, comprising the steps of:
a) soaking the corn kernels in water to produce soaked kernels;
b) grinding the soaked kernels to produce ground kernels;
c) separating germ from the ground kernels to produce a corn kernel mass comprising fiber, starch and gluten; and
d) subjecting the corn kernel mass, particularly a fine fiber fraction, to a fiber washing procedure separating starch and gluten from the fiber;

wherein at least a PDI or a Thioredoxin peptide is present/added before or during step d).

Embodiment [6]. The method of embodiment 4, further comprising the steps of:
e) separating the starch from the gluten; and optionally
f) washing the starch.

Embodiment [7]. The method of any of embodiments 1-6, wherein the PDI or Thioredoxin peptide is present/added during fiber wash in amounts at least 10 μg EP/g DS, at least 25 μg EP/g DS, at least 50 μg EP/g DS, at least 75 μg EP/g DS, at least 100 μg EP/g DS, at least 200 μg EP/g DS, at least 300 μg EP/g DS, at least 500 μg EP/g DS, such as in the range from 10 to 2000 μg EP/g DS, 25 to 1000 μg EP/g DS, 50 to 800 μg EP/g DS, 100 to 500 μg EP/g DS.

Embodiment [8]. The method of any of the embodiments 1-6, wherein SO₂ is present/added during fiber wash in amounts of at least 200 ppm, at least 300 ppm, at least 400 ppm, at least 450 ppm, at least 500 ppm, at least 600 ppm, at least 700 ppm, at least 800 ppm, such as e.g., in a range from 200-3000 ppm, 300-2000 ppm, 400-800 ppm.

Embodiment [9]. The method according to any of the preceding embodiments, wherein an effective amount of one or more hydrolytic enzymes are present/added before or during the fiber washing step, and wherein at least one of said hydrolytic enzymes is selected from xylanases and/or cellulases.

Embodiment [10]. The method of any of the preceding embodiments, wherein the xylanase is selected from the group consisting of a GH5 polypeptide, GH8 polypeptide, GH30 polypeptide, a GH10 polypeptide, a GH11 polypeptide, a GH8 polypeptide or a combination thereof.

Embodiment [11]. The method of any of the preceding embodiments, wherein the hydrolytic enzymes comprise one or more cellulases.

Embodiment [12]. The method of embodiment 10, wherein the cellulase(s) comprises one or more enzyme selected from the group consisting of an endoglucanase (EG), a cellobiohydrolase (CBH), and a beta-glucosidase.

Embodiment [13]. The method of embodiment 11, wherein the cellulase(s) comprises one or more enzyme selected from the group consisting of an endoglucanase, a cellobiohydrolase I, a cellobiohydrolase II, or a combination thereof.

Embodiment [14]. The method of embodiments 11-13, wherein the cellulases are derived from Trichoderma reesei.

Embodiment [15]. The method of any of the embodiments 11-13 where the cellulases are selected from the group consisting of: Aspergillus fumigatus GH10 xylanase, Aspergillus fumigatus beta-xylosidase, with a Trichoderma reesei cellulase preparation containing Aspergillus fumigatus cellobiohydrolase I of, Aspergillus fumigatus cellobiohydrolase II, Aspergillus fumigatus beta-glucosidase variant, and Penicillium sp. (emersonii) GH61 polypeptide.

Embodiment [16]. The method of embodiment 15, wherein the cellulases are selected from a composition comprising:
i) GH10 xylanase of SEQ ID NO 14, beta-xylosidase of SEQ ID NO: 15, cellobiohydrolase I of SEQ ID NO: 16, cellobiohydrolase II of SEQ ID NO: 17, beta-glucosidase of SEQ ID NO: 18, GH61 of SEQ ID NO: 19;
or
ii) a polypeptide having at least 90% identity, at least 95%, at least 98% to SEQ ID NO: 14, a polypeptide having at least 90% identity, at least 95%, at least 98% to SEQ ID NO: 15, a polypeptide having at least 90% identity, at least 95%, at least 98% to SEQ ID NO: 16, a polypeptide having at least 90% identity, at least 95%, at least 98% to SEQ ID NO: 17, a polypeptide having at least 90% identity, at least 95%, at least 98% to SEQ ID NO: 18, a polypeptide having at least 90% identity, at least 95%, at least 98% to SEQ ID NO: 19; and
iii) at least one endoglucanase derived from Trichoderma reesei.

Embodiment [17]. The method of any of the preceding embodiments, wherein the hydrolytic enzymes comprise an arabinofuranosidase.

Embodiment [18]. The method of embodiment 17, wherein the arabinofurasnosidase is selected from the group consisting of a GH43 polypeptide, a GH62 polypeptide, and a GH51 polypeptide.

Embodiment [19]. The method according to any of the preceding embodiments, wherein the corn kernel mass comprising fiber, starch and gluten from step c) is subjected to a further grinding step, preferably a fine grinding step before the fiber washing procedure in step d).

Embodiment [20]. The method according to any of the preceding embodiments, wherein said fiber washing procedure is performed with the use of a fiber washing system comprising a plurality of screen units (S1 . . . S4) being fluently connected in a counter current washing configuration; each screen unit being configured for separating a stream of corn kernel mass and liquid into two fractions: a first fraction (s) and a second fraction (f); said second fraction (f) containing a higher amount measured in wt % fiber than the first fraction (s); and optionally a space (V) arranged in the system and being fluently connected to receive one of said first fraction (s), one of said second fraction (f), or a mixed first and second fraction (s,f), preferably only a second fraction (f), and configured to provide an incubation time for one or both fractions received in the space; and outletting the thereby incubated one or both fractions to a downstream screen unit (S4),

wherein the system is configured for
inletting corn kernel mass and liquid to the most upstream screen unit (S1)
outletting the first fraction (s1) from the most upstream screen unit (S1) as a product stream containing starch,
inletting process water, preferably arranged for inletting process water to a most downstream screen unit (S4),
outletting the second fraction (f4) from most downstream screen unit (S4) as a washed corn kernel mass containing a lower amount of starch and gluten than the original corn kernel mass; and
optionally introducing hydrolytic enzymes into the system.

Embodiment [21]. The method according to embodiments 20, wherein said fiber washing procedure comprises the use of a fiber washing system comprising a space (V)/tank configured to provide a total retention time in the fiber washing system of at least 35 minutes and less than 48 hours.

Embodiment [22]. The method according to any of embodiments 20-21, wherein said space (V) has a volume which is in the range of 50-1000 m³.

Embodiment [23]. The method according to any of embodiments 20-22, wherein said space (V) has a volume which is in the range of 80 and 250 m³.

Embodiment [24]. The method according to any of the embodiments 20-23, wherein the incubation time in said space (V)/tank configured into the fiber washing system is at least 5 minutes and less than 48 hours, such as between 35 minutes and 24 hours, 35 minutes and hours, 35 minutes and 6 hours, 35 minutes and 5 hours, 35 minutes and 4 hours, 35 minutes and 3 hours, 35 minutes and 2 hours, 45 minutes and 48 hours, 45 minutes and 24 hours, 45 minutes and 12 hours, 45 minutes and 6 hours, 45 minutes and 5 hours, 45 minutes and 4 hours, 45 minutes and 3 hours, 45 minutes and 2 hours.

Embodiment [25]. The method according to any of the preceding embodiments, wherein the incubation temperature is between 25° C. and 95° C., such as between 25 and 90° C., 25 and 85° C., 25 and 80° C., 25 and 75° C., 25 and 70° C., 25 and 65° C., 25 and 60° C., 25 and 55° C., 25 and 53° C., 25 and 52° C., 30 and 90° C., 30 and 85° C., 30 and 80° C., 30 and 75° C., 30 and 70° C., 30 and 65° C., 30 and 60° C., 30 and 55° C., 30 and 53° C., 30 and 52° C., 35 and 90° C., 35 and 85° C., 35 and 80° C., 35 and 75° C., 35 and 70° C., 35 and 65° C., 35 and 60° C., 35 and 55° C., 35 and 53° C., 35 and 52° C., 39 and 90° C., 39 and 85° C., 39 and 80° C., 39 and 75° C., 39 and 70° C., 39 and 65° C., 39 and 60° C., 39 and 55° C., 39 and 53° C., 39 and 52° C., preferably 46 and 52° C.

Embodiment [26]. The method according to any of the preceding embodiments, wherein the one or more hydrolytic enzymes is expressed in an organism with a cellulase background, such as Trichoderma reesei.

Embodiment [27]. The method according to any of the preceding embodiments, wherein the effective amount of one or more hydrolytic enzymes admixed/contacted with one or more fractions of said ground corn kernel mass, is between 0.005-0.5 kg enzyme protein/metric tonne corn kernels entering the wet milling process.

Embodiment [28]. The method according to any of the preceding embodiments, wherein the source of SO₂ is selected from sodium metabisulfite (Na₂S₂O₅), NaHSO₃ and/or addition of SO₂ gas.

Embodiment [29]. The method according to any of the preceding embodiments, wherein the PDI or Thioredoxin peptide comprises at least one catalytic domain belonging to the thioredoxin superfamily of redox proteins, and wherein this domain comprises an active site characterized by having a CXXC motif.

Embodiment [30]. The method of embodiment 29, wherein the PDI comprises two catalytic domains, each comprising an active site characterized by having a CXXC motif, separated by at least one non-catalytic domain.

Embodiment [31]. The method of embodiments 29-30, wherein the CXXC motif is selected from the group consisting of CGHC, CTHC, CPHC, CGPC and CSMC, particularly CGHC or CGPC.

Embodiment [32]. The method of any of embodiments 29-31, wherein the catalytic domain belongs to family Pf00085.

Embodiment [33]. The method of embodiments 29-32, wherein the PDI comprises a three domain structure Pf00085-Pf13848-Pf00085 with a central Pf13848 domain flanked by two Pf00085 domains, one at the N-terminus and the other at the C-terminus.

Embodiment [34]. The method according to any of embodiments 1-33, wherein the PDI is selected from the group consisting of:
(a) a polypeptide having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 1;
(b) a variant of the mature polypeptide of SEQ ID NO: 1 comprising a substitution, deletion, and/or insertion at one or more (several) positions; and
(c) a fragment of the polypeptide of (a), or (b) that has PDI activity.

Embodiment [35]. The method according to any of embodiments 1-33, wherein the PDI is selected from the group consisting of:
(a) a polypeptide having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 2;
(b) a variant of the mature polypeptide of SEQ ID NO: 2 comprising a substitution, deletion, and/or insertion at one or more positions; and
(c) a fragment of the polypeptide of (a), or (b) that has PDI activity.

Embodiment [36]. The method according to any of embodiments 1-33, wherein the PDI is selected from the group consisting of:
(a) a polypeptide having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 3;
(b) a variant of the mature polypeptide of SEQ ID NO: 3 comprising a substitution, deletion, and/or insertion at one or more positions; and
(c) a fragment of the polypeptide of (a), or (b) that has PDI activity.

Embodiment [37]. The method according to any of embodiments 1-33, wherein the PDI is selected from the group consisting of:
(a) a polypeptide having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 4;
(b) a variant of the mature polypeptide of SEQ ID NO: 4 comprising a substitution, deletion, and/or insertion at one or more positions; and
(c) a fragment of the polypeptide of (a), or (b) that has PDI activity.

Embodiment [38]. The method according to any of embodiments 1-33, wherein the Thioredoxin peptide is selected from the group consisting of:
(a) a polypeptide having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 5;
(b) a variant of the mature polypeptide of SEQ ID NO: 5 comprising a substitution, deletion, and/or insertion at one or more positions; and
(c) a fragment of the polypeptide of (a), or (b) that has Thioredoxin peptide activity.

Embodiment [39]. The method according to any of the preceding embodiments wherein the xylanase is selected from the group consisting of:
(a) a polypeptide having at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide of SEQ ID NO: 8;
(b) a fragment of the polypeptide of (a), that has xylanase activity.

Embodiment [40]. The method according to any of the preceding embodiments wherein the xylanase is selected from the group consisting of:
(a) a polypeptide having at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 11;
(b) a fragment of the polypeptide of (a), that has xylanase activity.

Embodiment [41]. The method of any of the preceding embodiments, wherein the arabinofuranosidase is selected from the group consisting of:
(a) a polypeptide having at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 13;
(b) a fragment of the polypeptide of (a), that has arabinofuranosidase activity.

Embodiment [42]. The method according to any of the preceding embodiments wherein the xylanase is selected from the group consisting of:
(a) a polypeptide having at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 10;
(b) a fragment of the polypeptide of (a), that has xylanase activity.

Embodiment [43]. The method of any of the preceding embodiments, wherein the arabinofuranosidase is selected from the group consisting of:
(a) a polypeptide having at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 12;
(b) a fragment of the polypeptide of (a), that has arabinofuranosidase activity.

Embodiment [44]. The method according to any of the preceding embodiments, wherein the celullases are derived from Trichoderma reesei or Humicula insolens.

Embodiment [45]. The method according to any of the preceding embodiments, wherein the celullases are derived from Trichoderma reesei background cellulases having a CBH I and CBH II from Aspergillus fumigatus.

Embodiment [46]. An isolated or purified polypeptide having protein disulfide isomerase activity, selected from the group consisting of:
(a) a polypeptide having at least 85% sequence identity to SEQ ID NO: 1;
(b) a polypeptide having at least 85% sequence identity to amino acids 21-516 of SEQ ID NO: 1;
(c) a polypeptide having at least 85% sequence identity to a mature polypeptide of SEQ ID NO: 1;
(d) a polypeptide derived from a mature polypeptide of SEQ ID NO: 1 by substitution, deletion or addition of one or several amino acids in the mature polypeptide of SEQ ID NO: 1;
(e) a polypeptide encoded by a polynucleotide having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide encodimg sequence of SEQ ID NO: 20; and
(f) a fragment of the polypeptide of (a), (b), (c), (d), or (e) that has protein disulfide isomerase activity.

Embodiment [47]. The polypeptide of embodiment 46, having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1.

Embodiment [48]. The polypeptide of any one of embodiments 46-47, having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 1.

Embodiment [49]. The polypeptide of embodiment 46, comprising, consisting essentially of, or consisting of SEQ ID NO: 1 or a mature polypeptide thereof; or amino acids 21-516 of SEQ ID NO: 1.

Embodiment [50]. An isolated or purified polypeptide having protein disulfide isomerase activity, selected from the group consisting of:
(a) a polypeptide having at least 85% sequence identity to SEQ ID NO: 2;
(b) a polypeptide having at least 85% sequence identity to amino acids 21-513 of SEQ ID NO: 2;
(c) a polypeptide having at least 85% sequence identity to a mature polypeptide of SEQ ID NO: 2;
(d) a polypeptide derived from a mature polypeptide of SEQ ID NO: 2 by substitution, deletion or addition of one or several amino acids in the mature polypeptide of SEQ ID NO: 2;
(e) a polypeptide encoded by a polynucleotide having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide encodimg sequence of SEQ ID NO: 21; and
(f) a fragment of the polypeptide of (a), (b), (c), (d), or (e) that has protein disulfide isomerase activity.

Embodiment [51]. The polypeptide of embodiment 46, having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 2.

Embodiment [52]. The polypeptide of any one of embodiments 50-51, having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 2.

Embodiment [53]. The polypeptide of embodiment 50, comprising, consisting essentially of, or consisting of SEQ ID NO: 1 or a mature polypeptide thereof; or amino acids 21-513 of SEQ ID NO: 2.

Embodiment [54]. An isolated or purified polypeptide having protein disulfide isomerase activity, selected from the group consisting of:
(a) a polypeptide having at least 85% sequence identity to SEQ ID NO: 3;
(b) a polypeptide having at least 85% sequence identity to amino acids 21-516 of SEQ ID NO: 3;
(c) a polypeptide having at least 85% sequence identity to a mature polypeptide of SEQ ID NO: 3;
(d) a polypeptide derived from a mature polypeptide of SEQ ID NO: 3 by substitution, deletion or addition of one or several amino acids in the mature polypeptide of SEQ ID NO: 3;
(e) a polypeptide encoded by a polynucleotide having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide encodimg sequence of SEQ ID NO: 22; and
(f) a fragment of the polypeptide of (a), (b), (c), (d), or (e) that has protein disulfide isomerase activity.

Embodiment [55]. The polypeptide of embodiment 54, having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 3.

Embodiment [56]. The polypeptide of any one of embodiments 54-55, having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 3.

Embodiment [57]. The polypeptide of embodiment 54, comprising, consisting essentially of, or consisting of SEQ ID NO: 3 or a mature polypeptide thereof; or amino acids 21-516 of SEQ ID NO: 3.

Embodiment [58]. An isolated or purified polypeptide having protein disulfide isomerase activity, selected from the group consisting of:
(a) a polypeptide having at least 85% sequence identity to SEQ ID NO: 4;
(b) a polypeptide having at least 85% sequence identity to amino acids 21-517 of SEQ ID NO: 4;
(c) a polypeptide having at least 85% sequence identity to a mature polypeptide of SEQ ID NO: 4;
(d) a polypeptide derived from a mature polypeptide of SEQ ID NO: 4 by substitution, deletion or addition of one or several amino acids in the mature polypeptide of SEQ ID NO: 4;
(e) a polypeptide encoded by a polynucleotide having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide encodimg sequence of SEQ ID NO: 23; and
(f) a fragment of the polypeptide of (a), (b), (c), (d), or (e) that has protein disulfide isomerase activity.

Embodiment [59]. The polypeptide of embodiment 58, having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 4.

Embodiment [60]. The polypeptide of any one of embodiments 58-59, having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 4.

Embodiment [61]. The polypeptide of embodiment 58, comprising, consisting essentially of, or consisting of SEQ ID NO: 1 or a mature polypeptide thereof; or amino acids 21-517 of SEQ ID NO: 4.

Embodiment [62]. An isolated or purified thioredoxin polypeptide having the ability to reduce protein disulfide bonds, selected from the group consisting of:
(a) a polypeptide having at least 85% sequence identity to SEQ ID NO: 5;
(b) a polypeptide having at least 85% sequence identity to amino acids 1-110 of SEQ ID NO: 5;
(c) a polypeptide having at least 85% sequence identity to a mature polypeptide of SEQ ID NO: 5;
(d) a polypeptide derived from a mature polypeptide of SEQ ID NO: 5 by substitution, deletion or addition of one or several amino acids in the mature polypeptide of SEQ ID NO: 5;
(e) a polypeptide encoded by a polynucleotide having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide encodimg sequence of SEQ ID NO: 24; and
(f) a fragment of the polypeptide of (a), (b), (c), (d), or (e) that has thioredoxin activity activity.

Embodiment [63]. The polypeptide of embodiment 62, having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 5.

Embodiment [64]. The polypeptide of any one of embodiments 62-63, having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 5.

Embodiment [65]. The polypeptide of embodiment 62, comprising, consisting essentially of, or consisting of SEQ ID NO: 5 or a mature polypeptide thereof; or amino acids 1-110 of SEQ ID NO: 5.

Embodiment [66]. The polypeptide of any of embodiments 46-65, wherein the amount of starch and/or gluten, particularly insoluble starch and gluten, released from a fiber rich fraction of ground kernels during the wet milling process is increased compared to a process where no PDI or Thioredoxin peptide is present/added in a fiber-washing step.

Embodiment [67]. A composition comprising the polypeptide of any one of embodiments 46-66.

Embodiment [68]. The composition of embodiment 67, further comprising a xylanase of embodiment 10 and/or a cellulase of embodiments 11-16.

Embodiment [69]. A whole broth formulation or cell culture composition comprising the polypeptide of any one of embodiments 46-66.

Embodiment [70]. An isolated or purified polynucleotide encoding the polypeptide of any one of embodiments 46-66.

Embodiment [71]. A nucleic acid construct or expression vector comprising the polynucleotide of embodiment 70, wherein the polynucleotide is operably linked to one or more heterologous control sequences that direct the production of the polypeptide in an expression host.

Embodiment [72]. A recombinant host cell comprising the polynucleotide of embodiment 70 operably linked to one or more heterologous control sequences that direct the production of the polypeptide.

Embodiment [73]. A method of producing a polypeptide having Protein disulfide isomerase activity or thioredoxin peptide activity, comprising cultivating the recombinant host cell of embodiment 72 under conditions conducive for production of the polypeptide, and optionally recovering the polypeptide.

### Examples

### Assays

Insulin Reduction Assay with Dithiothreitol

### Principle

The two cystines in bovine insulin are reduced by PDI in the presence of dithiothreitol (DTT).

The reaction is followed by analyzing disappearance of intact insulin and formation of the two peptides by matrix assisted laser desorption ionization mass spectrometry (MALDI MS).

### Chemicals

Ammonium bicarbonate (ABC), Fluka 09830
Ethylenediamine tetraacetic acid (EDTA), di-sodium salt, Merck 1.08418
Ammonium acetate, Sigma-Aldrich 32301
Dithiothreitol (DTT), Sigma D0632
Insulin from bovine pancreas, Sigma 15500
Trifluoroacetic acid (TFA), Sigma 302031
2′,5′-dihydroxyacetophenone (DHAP), Sigma D107603
Acetonitrile, Sigma 34888

### Reagents

ABC buffer: 10 mM ammonium bicarbonate, pH 7
EDTA solution: 100 mM EDTA, pH 8
Acetate buffer: 25 mM ammonium acetate, pH 5
Substrate stock: 10 mg/mL insulin in ABC buffer
Substrate working solution: 100 μL substrate stock+20 μL EDTA solution+780 μL 25 mM
acetate buffer. After addition of EDTA, wait until solution is clear before adding acetate buffer
DTT solution: 200 mM DTT in Milli Q water
Stop reagent: 2% (V/V) TFA in Milli Q water
Matrix: 20 mg/mL DHAP in acetonitrile

### Materials

MALDI MS: Bruker UltrafleXtreme™
Bruker AnchorChip MALDI target plate
Biosan Plate Thermoshaker PST 100-HL

### Enzymes

Protein disulfide isomerase (PDI)

### Procedure

1) Prepare enzyme working solutions (typically 0-100 μg enzyme protein/mL resulting in 0-10 ppm in-assay) and a freshly prepared substrate working solution
2) 90 μL substrate working solution and 10 μL enzyme working solution is mixed in a 96 well microtiter plate. For the enzyme blank, 10 μL Milli Q water is added instead of the enzyme solution.
3) Reaction is started by addition of 1 μL DTT solution or for the blank 1 μL Milli Q water (reagent blank)
4) Plate is incubated for 10 minutes at 40° C., shaking at 550 rpm
5) After incubation, reaction is stopped by adding 100 μL Stop reagent
6) For the MALDI MS analysis, 2 μL sample is mixed with 4 μL DHAP, and 0.7 μL is transferred to target plate. Mass spectra is obtained in positive, linear mode. Ion Source 1 and 2 set to 20 kV and 18.8 kV, respectively, a delay of 11340 ns and a sampling rate of 6.4 ns. Each spectrum is the cumulative average of 5000 shots per sample spot and the ion mass range set to 500-7000.
7) The activity of the enzyme can be observed as a decrease in the mass peak area of the intact bovine insulin (mass range 5670-6000) and an increase in the appearance of the two A and B peptide chains at mass range 2300-2450 and 3325-3500, respectively, compared to the spectra of the enzyme blank and the reagent blank. (It should be noted that peaks corresponding to several sodium adducts of both the intact insulin and the two peptides can be observed, these are included in the mass ranges used.)
8) If required, a standard curve can be prepared by running the assay with different enzyme concentrations. Calculate the ratio between the ion peak area of chain A (or B) and the ion peak area for the ions for the intact bovine insulin and plot the ratio against enzyme concentration.
   Insulin Reduction Assay with Sulfite

### Principle

The two cystines in bovine insulin are reduced by PDI in the presence of sulfite. The reaction is followed by analyzing disappearance of intact insulin and formation of the two peptides by matrix assisted laser desorption ionization mass spectrometry (MALDI MS).

### Chemicals

Ammonium bicarbonate (ABC), Fluka 09830
Ethylenediamine tetraacetic acid (EDTA), di-sodium salt, Merck 1.08418
Ammonium acetate, Sigma-Aldrich 32301
Sodium metabisulfite, Merck 31448
Insulin from bovine pancreas, Sigma 15500
Trifluoroacetic acid (TFA), Sigma 302031
2′,5′-dihydroxyacetophenone (DHAP), Sigma D107603
Acetonitrile, Sigma 34888

### Reagents

ABC buffer: 10 mM ammonium bicarbonate, pH 7
EDTA solution: 100 mM EDTA, pH 8
Acetate buffer: 25 mM ammonium acetate, pH 5
Substrate stock: 10 mg/mL insulin in ABC buffer
Substrate working solution: 100 μL substrate stock+20 μL EDTA solution+780 μL 25 mM
acetate buffer. After addition of EDTA, wait until solution is clear before adding acetate buffer
Sulfite solution: 60 mM sodium metabisulfite in Milli Q water
Stop reagent: 2% (V/V) TFA in Milli Q water
Matrix: 20 mg/mL DHAP in acetonitrile

### Materials

MALDI MS: Bruker UltrafleXtreme™
Bruker AnchorChip MALDI target plate
Biosan Plate Thermoshaker PST 100-HL

### Enzymes

Protein disulfide isomerase (PDI) (Thermaascus crustaceus (P73RVY)

### Procedure

1) Prepare enzyme working solutions (typically 0-100 μg enzyme protein/mL resulting in 0-10 ppm in-assay) and a freshly prepared substrate working solution and a freshly prepared sulfite solution.
2) 90 μL substrate working solution and 10 μL enzyme working solution is mixed in a 96 well microtiter plate. For the enzyme blank, 10 μL Milli Q water is added instead of the enzyme solution.
3) Reaction is started by addition of 5 μL sulfite solution or for the blank 5 μL Milli Q water (reagent blank)
4) Plate is incubated for 30 minutes at 40° C., shaking at 550 rpm
5) After incubation, reaction is stopped by adding 100 μL Stop reagent
6) For the MALDI MS analysis, 2 μL sample is mixed with 4 μL DHAP, and 0.7 μL is transferred to target plate. Mass spectra is obtained in positive, linear mode. Ion Source 1 and 2 set to 20 kV and 18.8 kV, respectively, a delay of 11340 ns and a sampling rate of 6.4 ns. Each spectrum is the cumulative average of 5000 shots per sample spot and the ion mass range set to 500-7000.
7) The activity of the enzyme can be observed as a decrease in the mass peak area of the intact bovine insulin (mass range 5670-6000) and an increase in the appearance of the two A and B peptide chains at mass range 2300-2640 and 3325-3630, respectively, compared to the spectra of the enzyme blank and the reagent blank. (It should be noted that peaks corresponding to several sodium adducts of both the intact insulin and the two peptides can be observed, these are included in the mass ranges used. In addition, the formed A and B peptide chain is present as a mix of a reduced form and a form with added SO₃-residue, both forms are included in the mass range set.)
8) If required, a standard curve can be prepared by running the assay with different enzyme concentrations. Calculate the ratio between the ion peak area of chain A (or B) and the ion peak area for the ions for the intact bovine insulin and plot the ratio against enzyme concentration.

### Strains

The strain Thermoascus aurantiacus was isolated from soil, Yunnan, China in 1998. The strain Thermoascus crustaceus was isolated from a plant, California, USA, (CBS181.67). Strains were identified and taxonomy was assigned based on DNA sequencing of the ITS (Table 1).

TABLE 1

| SEQ ID NO | Organism_name | Source country |
|---|---|---|
| 1 | Thermoascus crustaceus | USA |
| 2 | Keithomyces carneus | China |
| 3 | Aspergillus spinulosporus | China |
| 4 | Thermoascus aurantiacus | China |
| 5 | Aspergillus aculeatus | CBS101.43 |

### Example 1: Cloning of Protein Disulfide Isomerase from Thermoascus aurantiacus (SEQ ID NO: 4)

The protein disulfide isomerase with nucleotide sequence SEQ ID NO: 7 was PCR amplified from genomic DNA isolated from Thermoascus aurantiacus and cloned into the expression vector pDAU724 (WO2018/113745).

The final expression plasmid was transformed into the Aspergillus oryzae DAU785 expression host (WO95/002043). 100 μl of protoplasts were mixed with 2.5-10 μg of the expression plasmid comprising the protein disulfide isomerase gene and 300 μl of 60% PEG 4000, 10 mM CaCl₂, and 10 mM Tris-HCl pH7.5 and gently mixed. The mixture was incubated at room temperature for 30 minutes and the protoplasts were spread onto sucrose plates supplemented with 100 mM sodium nitrate for selection.

One recombinant A. oryzae clone containing the protein disulfide isomerase expression construct was selected and cultivated in 2400 ml YPM (1% Yeast extract, 2% Peptone and 2% Maltose) in shake flasks for 3 days at 30° C. under 80 rpm agitation. Enzyme containing supernatants were harvested by filtration using a 0.22 μm 1-liter bottle top vacuum filter (Thermo Fisher Scientific Inc., Waltham, MA, USA).

### Example 2: Cloning of Protein Disulfide Isomerase from Thermoascus crustaceus (SEQ ID NO: 1)

The protein disulfide isomerase with nucleotide sequence SEQ ID NO: 6 was PCR amplified from genomic DNA isolated from Thermoascus crustaceus and cloned into the expression vector pDAU724 (WO2018/113745).

The final expression plasmid was transformed into the Aspergillus oryzae DAU785 expression host (WO95/002043). 100 μl of protoplasts were mixed with 2.5-10 μg of the expression plasmid comprising the protein disulfide isomerase gene and 300 μl of 60% PEG 4000, 10 mM CaCl₂, and 10 mM Tris-HCl pH7.5 and gently mixed. The mixture was incubated at room temperature for 30 minutes and the protoplasts were spread onto sucrose plates supplemented with 100 mM sodium nitrate for selection.

One recombinant A. oryzae clone containing the protein disulfide isomerase expression construct was selected and cultivated in 2400 ml YPM (1% Yeast extract, 2% Peptone and 2% Maltose) in shake flasks for 3 days at 30° C. under 80 rpm agitation. Enzyme containing supernatants were harvested by filtration using a 0.22 μm 1-liter bottle top vacuum filter (Thermo Fisher Scientific Inc., Waltham, MA, USA).

### Example 3: Effect of Adding PDI in Fiber-Wash

Coarse pericarp fiber after the fiber press in a corn wet-milling process was used as substrate and was incubated with enzymes at process relevant conditions (i.e. pH 4-5, 40-48° C. and 400 ppm sulfur dioxide). After incubation, the fiber was vacuum filtered through a 60 μm pore size filter. The fiber cake was resuspended in water and filtered a second time. The extracted solid protein and starch was collected and washed thoroughly by centrifugation. The amount of protein in the resulting pellet containing the extracted starch and gluten from the collected filtrates was determined by measurement of total nitrogen by LECO and using a nitrogen-to-protein conversion factor of 6.25 (Jones, USDA Circular No 183, 1931) and normalized to the weight of the starting fiber dry solids.

### Chemicals

Coarse pericarp fiber sample
Sodium metabisulfite, Merck art. 31448
Sodium acetate, Sigma Aldrich, S8625

### Reagents

Sulfite: Hydrogen sulfite is generated by adding sodium metabisulfite (Na₂S₂O₅) into the buffer following the reaction of Na₂S₂O₅+H₂O->2Na⁺+2HSO₃·400 ppm SO₂was thus prepared from 593.6 mg/L metabisulfite.
Buffer: 0.1M sodium acetate buffer, pH 4.0

### Materials

Infors-HT Multitron Pro incubation shaker
Avanti J-E centrifuge
Millipore steriflip tube top filter unit, 60 μm pore size (Merck, order no. SCNY0060)
EZ-2 Elite Solvent Evaporator
LECO Nitrogen Determinator FP628

### Enzymes

PDI A: Protein disulfide isomerase from Thermoascus crustaceus, disclosed as SEQ ID NO: 1
PDI B: Protein disulfide isomerase derived from Keithomyces carneus, disclosed as SEQ ID NO: 2
PDI C: Protein disulfide isomerase derived from Aspergillus spinulosporus, disclosed as SEQ ID NO: 3
PDI D: Protein disulfide isomerase derived from Thermoascus aurantiacus, disclosed as SEQ ID NO: 4
Trx thioredoxinThioredoxin peptide derived from Aspergillus aculeatus, disclosed as SEQ ID NO: 5
GH5 Xylanase A: GH5 xylanase derived from Chryseobacterium sp-10696 and disclosed as SEQ ID NO: 8
GH10 Xylanase B: GH10 xylanase derived from Aspergillus niger and disclosed as SEQ ID NO: 10
GH62 Arabinofuranosidase A: GH62 arabinofuranosidase derived from Aspergillus niger and disclosed as SEQ ID NO: 12
Cellulase A/Celluclast 1.5 L: A cellulase mix derived from Trichoderma reesei. This cellulase composition will comprise all cellulase activities expressed in T. reesei; e.g., endoglucanases, and cellobiohydrolases. Commercially available from Sigma Aldrich, Celluclast 1.5 L

### Procedure for 15 mL Fiber Washing Assay

1. The coarse fiber sample was suspended in buffer (pH 4-5, 0.02M sodium acetate final concentration) to 15 mL slurry containing 5% dry solids. Amount of total fiber dry weight was recorded (weight_{fiber}). (The exact pH is specified in the examples)
2. To this slurry enzyme was added a mix of xylanase and cellulase (the exact amounts of enzymes is specified in the examples), and 400 ppm SO₂ (final concentration).
3. Samples were incubated for 4 hours at 600 rpm in an incubation shaker at temperature from 40-48° C. (exact temperature specified in the examples).
4. After incubation the fibers were vacuum filtered in Millipore Steriflip filter unit
5. The retained fibers were resuspended to a volume of 20 mL with distilled water, thoroughly vortexted and vacuum filtered a second time.
6. The two filtrates, containing the extracted starch and gluten, were pooled.
7. The combined filtrates from (6) were centrifuged (3,500 rpm, 20 minutes)
8. After centrifugation, the supernatant was slowly removed using a 50 mL serological pipette as to not disturb the starch and gluten pellet. Steps five through 8 were repeated once more.
9. The pellet from (8) was washed twice using 20 mL distilled water in each wash step to remove solubilized components.
10. After washing and final removal of the supernatant a total volume of 10 mL remained, incl. pellet. The excess water was removed using Solvent Evaporator (method: aqueous, maximum 62° C., 2 hours, 3,000 rpm, 5 mbar).
11. Upon cooling the sample was weighed (weightₚₑₗₗₑₜ) and vigorously vortexed to achieve homogeneity.
12. 170 μL of the slurry from (11) was pipetted using wide bore tips into a LECO tin capsule (NC9804300, Fisher Scientific) and the exact weight recorded (weight_{slurry}).
13. The samples were analyzed for total nitrogen (Tot_{N}) using a LECO FP628 and a nitrogen-to-protein factor of 6.25 (Jones, USDA Circular No 183, 1931) was used to convert to percent protein of sample.
14. The total amount of protein release was calculated using the percent protein and final weight of the pellet. The grams of protein released is then normalized back to starting grams of fiber dry solids.

### Calculation

$$Protein concentration in slurry ( Conc . ) = \frac{{Tot}_{N} * 6.25}{{weight}_{slurry}}$$

$$Protein extraction yield = \frac{Conc * {weight}_{pellet}}{{weight}_{fiber}} \%$$

The performance of Protein disulfide isomerase from Thermoascus crustaceus, SEQ ID NO: 1, was tested using the 15 mL fiber washing assay with experimental conditions shown in Table 2. The amount of protein released is shown in Table 3. The results are based on 3 separate experiments at each condition

TABLE 2
Experimental conditions

| | | | | Xylanase | | PDI |
|---|---|---|---|---|---|---|
| | | | Sulfur | A (seq | | A (seq |
| | Temp. | | Dioxide | id no: 8) | Cellulase A | id no: 1) |
| | (° C.) | pH | (ppm) | (μg/gDS) | (μg/gDS) | (μg/gDS) |
| | 48 | 4 | 400 | 100 | 900 | 500 |
| Control | 48 | 4 | 400 | 100 | 900 | — |

TABLE 3
Protein extraction yields

| | Protein Release |
|---|---|
| Treatment | (mg/gDS) |
| Control | 31.0 ± 6 |
| PDI, seq. id no: 1 | 41.1 ± 7 |

Results are shown as mean±standard deviation, n=3

The performance of Protein disulfide isomerase from Keithomyces carneus, SEQ ID NO: 2, was tested using the 15 mL fiber washing assay with experimental conditions shown in Table 4. The amount of protein released is shown in Table 5. The results are based on 3 separate experiments at each condition.

TABLE 4
Experimental conditions

| | | | | Xylanase | | PDI |
|---|---|---|---|---|---|---|
| | | | Sulfur | A (seq | | B (seq |
| | Temp. | | Dioxide | id no: 8) | Cellulase A | id no: 2) |
| | (° C.) | pH | (ppm) | (μg/gDS) | (μg/gDS) | (μg/gDS) |
| | 40 | 5 | 400 | 100 | 900 | 300 |
| Control | 40 | 5 | 400 | 100 | 900 | — |

TABLE 5
Protein extraction yields

| | Protein Release |
|---|---|
| Treatment | (mg/gDS) |
| Control | 40.2 ± 3 |
| PDI, seq. id no: 2 | 45.9 ± 3 |

Results are shown as mean±standard deviation, n=3

### Example 4

### Enzymes:

Protein disulfide isomerase A: Protein disulfide isomerase derived from Thermoascus crustaceus SED ID NO: 1
GH10 Xylanase B: GH10 xylanase derived from Aspergillus niger SEQ ID NO: 10
GH62 Arabinofuranosidase A: GH62 arabinofuranosidase derived from Aspergillus niger SEQ ID NO: 12
Cellulase A: Cellulase mix derived from Trichoderma reesei. This cellulase composition will comprise all cellulase activities expressed in T. reesei; e.g., endoglucanases, and cellobiohydrolases. Commercially available from Sigma Aldrich, Celluclast 1.5 L

0.5-g medium throughput (MTP) fiber assay (15 mL Fiber Washing Assay) was performed with 5% fiber dry substance incubating at pH4.0 in 20 mM sodium acetate buffer with 400 ppm hydrogen sulfite (HSO₃⁻), 48° C. for 120 minutes at dose of 1000 μg enzyme protein per gram fiber dry substance, using a blend including Cellulase A, GH10 Xylanase B and GH62 Arabinofuranosidase A, in combination with Protein disulfide isomerase A. Blend consists of 30% Protein disulfide isomerase A, 10.5% of GH10 Xylanase B, 3.5% of GH62 Arabinofuranosidase A and the remaining 56% from Cellulase A based on enzyme protein. Hydrogen sulfite is generated by adding sodium metabisulfite (Na₂S₂O₅) into the buffer following the reaction of Na₂S₂O₅+H₂O->2Na⁺+2HSO₃⁻. For comparison, blend containing 80% Cellulase A, 15% GH10 Xylanase A and 5% GH62 Arabinofuranosidase A only (without Protein disulfide isomerase A) at both low dose (700 μg EP/g-ds fiber) and high dose (1000 μg EP/g-ds fiber) were included. The corn fiber with 16.79% residual starch and 10.00% residual protein was used as substrate in the MTP fiber assay. Release of starch+gluten (dry substance) as well as individual protein from corn fiber at the specified treatment below was measured.

TABLE 6

| | | A | B | C | D |
|---|---|---|---|---|---|
| | | μg enzyme protein/g | | | |
| | Experiment | fiber (dry substance) | | | |
| Enzymes | Cellulase A | — | 560 | 800 | 560 |
| added | GH10 Xylanase B | — | 105 | 150 | 105 |
| | GH62 Arabinofu- | — | 35 | 50 | 35 |
| | ranosidase A | | | | |
| | Protein Disulfide | — | — | — | 300 |
| | Isomerase A | | | | |
| | Total enzyme | — | 700 | 1000 | 1000 |
| | protein dosed | | | | |
| | Starch + Gluten | 10.0% | 14.5% | 17.1% | 17.5% |
| | Recovered | | | | |
| | Individual | 1.4% | 2.4% | 2.9% | 3.1% |
| | Protein | | | | |
| | Recovered | | | | |

Therefore, the addition of Protein disulfide isomerase A on top of Cellulase A+GH10 Xylanase B+GH62 Arabinofuranosidase A can significantly increase the yield of starch+gluten as well as protein in corn wet-milling process.

### Example 5 Increased Protein Release by Using Protein Disulfide Isomerase in Fiber Wash

The 15 mL fiber washing assay is performed over two experiments with conditions in Table 7. All treatments received a blend of Trichoderma reesei cellulase and xylanase A. The ratio of Trichoderma reesei cellulase to xylanase A was approximately 90:10 on a milligram enzyme protein basis. Each protein disulfide isomerase (PDI) was added to the appropriate treatments, while controls contained only cellulase and xylanase A. Total nitrogen was determined using a LECO FP628 and normalized to starting grams of fiber dry solids (gDS). The PDI diversity tested is in Table 2, and results of protein release over several experiments are in Table 3.

TABLE 7
Experimental conditions

| | | | | cellulase + | Protein |
|---|---|---|---|---|---|
| | Temper- | | Sulfur | xylanase mix | Disulfide |
| | ature | | Dioxide | Dose | Isomerase |
| | (° C.) | pH | (ppm) | (μg/gDS) | Dose (μg/gDS) |
| Experiment 1 | 48 | 4 | 400 | 700 | 500 |
| Experiment 2 | 40 | 5 | 400 | 700 | 300 |
| Experiment 3 | 48 | 5 | 400 | 700 | 300 |

TABLE 8
Protein disulfide isomerase diversity evaluated

| Donor | Activity | SEQ ID NO |
|---|---|---|
| Thermoascus crustaceus | PDI | SEQ ID NO: 1 |
| Keithomyces carneus | PDI | SEQ ID NO: 2 |
| Aspergillus | PDI | SEQ ID NO: 3 |
| spinulosporus | | |
| Thermoascus | PDI | SEQ ID NO: 4 |
| aurantiacus | | |

TABLE 9
Results from 15 mL fiber assay

| | Treatment | Protein Release (mg/gDS) |
|---|---|---|
| Experiment 1 | Control | 31.0 (6.1) |
| | SEQ ID NO: 1 | 41.1 (7.3) |
| Experiment 2 | Control | 40.2 (3.5) |
| | SEQ ID NO: 2 | 45.9 (3.1) |
| | SEQ ID NO: 4 | 42.3 (3.5) |
| Experiment 3 | Control | 45.3 (0.8) |
| | SEQ ID NO: 3 | 47.6 (0.1) |

(±standard deviation, n = 3)

### Example 6 Effectiveness of Single Thioredoxin Domain at Increasing Protein Yield from Corn Fiber in a Large Scale Fiber Washing Assay

Large scale corn fiber washing assay (5 gram assay): A 5 gram corn fiber assay generally includes incubating wet fiber samples obtained from a wet-milling plant, in the presence of enzymes at conditions relevant to the process (pH 3.5 to 4, Temp around 48-52° C.) and over a time period of between 1 to 4 hr. After incubation, the fiber was transferred and pressed over a 75 micron screen or smaller, where the filtrates consist mainly of the separated starch and gluten. The washing process was repeated several times over the screen, and the washings were collected together with the initial filtrate. The collected filtrate were allowed to settle overnight before the supernatant was aspirated via vacuum. The remaining filtrate and insolubles were poured into 50 mL tubes and centrifuged in an Avanti J-E at 3,500 rpm for 10 minutes to pellet the starch and gluten. The supernatant was decanted and remaining pellet freeze dried overnight in a Labconco Freeze Drier or until all moisture was removed. Samples were ground using a SPEX SamplePrep Genogrinder at 1750 rpm for one minute. 100 to 150 milligrams of sample was weighed recorded. Samples were run for total nitrogen using a LECO FP628 and nitrogen-to-protein factor of 6.25 was used to convert to percent protein of sample (Jones, D. B. (1931). Factors for converting percentage of nitrogen in foods and feeds into percentages of proteins. USDA Circular, 183, 1-22.). The total amount of protein release was calculated using the percent protein and final weight of the pellet. The grams of protein released was then normalized back to starting grams of fiber dry solids.

The 5 gram fiber assay was performed at pH 4, incubating fiber at 48° C. for 2 hours with 400 ppm of sulfur dioxide. All treatments received an enzyme composition, which is composed of a blend of Trichoderma reesei cellulase and GH5 Xylanase A. The ratio of Trichoderma reesei to GH5 Xylanase A was approximately 90:10 on a milligram enzyme protein basis. Each experimental enzyme, SEQ ID NO: 1 or 5, was added to the appropriate treatments, while controls contained only the blend of Trichoderma reesei cellulase and GH5 Xylanase A. Total nitrogen was determined using a LECO FP628 and normalized to starting grams of fiber dry solids (gDS). The PDI diversity tested is in Table 10, and results of protein release over several experiments are in Table 11.

TABLE 10
Protein disulfide isomerase and thioredoxin diversity evaluated

| Donor | Activity | SEQ ID NO |
|---|---|---|
| Thermoascus crustaceus | PDI | SEQ ID NO: 1 |
| Aspergillus aculeatus | Trx | SEQ ID NO: 5 |

TABLE 11
Results from 15 mL fiber assay

| | Protein Release |
|---|---|
| Treatment | (mg/gDS) |
| Control | 19.1 (1.5) |
| SEQ ID NO: 1 | 20.3 (1.2) |
| SEQ ID NO: 5 | 20.9 (2.2) |

(±standard deviation, n = 3)

### Example 7

The activity of PDI from Themoascus crustaceus (SEQ ID NO: 1) was run as described in the Insulin activity assay with 0 to 10 μg/mL PDI (in-assay concentration) and 2 mM DTT. The resulting mass peak areas of the A peptide chain to intact insulin and mass peak areas of the B peptide chain to intact insulin are shown in table 12.

TABLE 12

| | Ratio | Ratio | Ratio | Ratio |
|---|---|---|---|---|
| | (A chain) | (B chain | (A chain) | (B chain |
| DTT conc. | 2 | 2 | 0 | 0 |
| (mM in assay) | | | | |
| Enzyme conc. | | | | |
| (μg/mL in assay) | | | | |
| 0 | 0.003 | 0.008 | 0.005 | 0.009 |
| 2.5 | 0.035 | 0.109 | 0.006 | 0.010 |
| 5 | 0.070 | 0.185 | 0.005 | 0.009 |
| 10 | 0.172 | 0.335 | 0.003 | 0.007 |

In the absence of either enzyme or DTT, a very small amount of the cystines in bovine insulin is reduced, whereas in the presence of the PDI, a significant amount of the cystines are reduced resulting in separation of the insulin into the A peptide and B peptide.

### Example 8

The activity of PDI from Thermoascus crustaceus (SEQ ID NO: 1) was run as described in the Insulin activity assay with 0 to 10 μg/mL PDI (in-assay concentration) and 2.9 mM sulfite. The resulting mass peak areas of the A peptide chain to intact insulin and mass peak areas of the B peptide chain to intact insulin are shown in table 13.

TABLE 13

| | Ratio | Ratio | Ratio | Ratio |
|---|---|---|---|---|
| | (A chain) | (B chain | (A chain) | (B chain |
| Sulfite conc. | 2.9 | 2.9 | 0 | 0 |
| (mM in assay) | | | | |
| Enzyme conc. | | | | |
| (μg/mL in assay) | | | | |
| 0 | 0.018 | 0.040 | 0.008 | 0.014 |
| 2.5 | 0.027 | 0.055 | 0.009 | 0.019 |
| 5 | 0.050 | 0.083 | 0.007 | 0.018 |
| 10 | 0.104 | 0.170 | 0.008 | 0.017 |

In the absence of either enzyme or sulfite, a very small amount of the cystines in bovine insulin is reduced, whereas in the presence of the PDI, a significant amount of the cystines are reduced resulting in separation of the insulin into the A peptide and B peptide.

1-73. (canceled)

74. A method for increasing starch yield and/or gluten yield from corn kernels in a wet milling process, the method comprising contacting ground a fiber rich fraction of ground corn kernels with an effective amount of a polypeptide that catalyzes changes in protein disulfide bonds selected from the group consisting of oxidation, reduction, and isomerization.

75. The method of claim 74, wherein the polypeptide is a Protein Disulfide Isomerase (PDI) (EC 5.3.4.1) or Thioredoxin peptide.

76. The method according to claim 75, wherein the PDI or Thioredoxin peptide is present/added to a fiber fraction.

77. The method according to claim 75, wherein the amount of insoluble starch and gluten, released from ground kernels during the wet milling process is increased compared to a process where no PDI or Thioredoxin peptide is present/added.

78. The method according to claim 77, wherein the increase measured as mg protein released per gram dry solids (mg/gDS) is at least 0.5% points, at least 0.75% points, at least 1.0% points, at least 1.5% points, at least 2.5% points, such as at least 5.0% points, compared to no addition of PDI, wherein fiber-wash is performed at a pH in the range from 3.5-5.5, and an enzyme dosage of at least 300 μg EP/gDS.

79. The method according to claim 74, comprising the steps of:
a) soaking the corn kernels in water to produce soaked kernels;
b) grinding the soaked kernels to produce ground kernels;
c) separating germ from the ground kernels to produce a corn kernel mass comprising fiber, starch and gluten; and
d) subjecting a fine fiber fraction of corn kernel mass to a fiber washing procedure separating starch and gluten from the fiber;
wherein at least a PDI or a Thioredoxin peptide is present/added before or during step d).

80. The method according to claim 79, wherein the PDI is selected from the group consisting of:
(a) a polypeptide having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 1, SEQ ID NO: 2, SEQ ID NO: 3, or SEQ ID NO: 4;
(b) a variant of the mature polypeptide of SEQ ID NO: 1, SEQ ID NO: 2, SEQ ID NO: 3, or SEQ ID NO: 4 comprising a substitution, deletion, and/or insertion at one or more (several) positions; and
(c) a fragment of the polypeptide of (a), or (b) that has PDI activity.

81. The method according to claim 79, wherein the Thioredoxin peptide is selected from the group consisting of:
(a) a polypeptide having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 5;
(b) a variant of the mature polypeptide of SEQ ID NO: 5 comprising a substitution, deletion, and/or insertion at one or more positions; and
(c) a fragment of the polypeptide of (a), or (b) that has Thioredoxin peptide activity.

82. An isolated or purified polypeptide having protein disulfide isomerase activity, selected from the group consisting of:
(a) a polypeptide having at least 85% sequence identity to SEQ ID NO: 1, SEQ ID NO: 2, SEQ ID NO: 3, or SEQ ID NO: 4;
(b) a polypeptide having at least 85% sequence identity to amino acids 21-516 of SEQ ID NO: 1, amino acids 21-513 of SEQ ID NO: 2, amino acids 21-516 of SEQ ID NO: 3, or amino acids 21-517 of SEQ ID NO: 4;
(c) a polypeptide having at least 85% sequence identity to a mature polypeptide of SEQ ID NO: 1, SEQ ID NO: 2, SEQ ID NO: 3, or SEQ ID NO: 4;
(d) a polypeptide derived from a mature polypeptide of SEQ ID NO: 1, SEQ ID NO: 2, SEQ ID NO: 3, or SEQ ID NO: 4 by substitution, deletion or addition of one or several amino acids in the mature polypeptide of SEQ ID NO: 1, SEQ ID NO: 2, SEQ ID NO: 3, or SEQ ID NO: 4;
(e) a polypeptide encoded by a polynucleotide having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide encoding sequence of SEQ ID NO: 20, SEQ ID NO 21, SEQ ID NO: 22, or SEQ ID NO: 23; and
(f) a fragment of the polypeptide of (a), (b), (c), (d), or (e) that has protein disulfide isomerase activity.

83. A composition comprising the polypeptide of claim 82.

84. An isolated or purified polynucleotide encoding the polypeptide of claim 82.

85. A nucleic acid construct or expression vector comprising the polynucleotide of claim 84, wherein the polynucleotide is operably linked to one or more heterologous control sequences that direct the production of the polypeptide in an expression host.

86. A recombinant host cell comprising the polynucleotide of claim 84 operably linked to one or more heterologous control sequences that direct the production of the polypeptide.

87. A method of producing a polypeptide having Protein disulfide isomerase activity, comprising cultivating the recombinant host cell of claim 86 under conditions conducive for production of the polypeptide, and optionally recovering the polypeptide.

88. An isolated or purified thioredoxin polypeptide having the ability to reduce protein disulfide bonds, selected from the group consisting of:
(a) a polypeptide having at least 85% sequence identity to SEQ ID NO: 5;
(b) a polypeptide having at least 85% sequence identity to amino acids 1-110 of SEQ ID NO: 5;
(c) a polypeptide having at least 85% sequence identity to a mature polypeptide of SEQ ID NO: 5;
(d) a polypeptide derived from a mature polypeptide of SEQ ID NO: 5 by substitution, deletion or addition of one or several amino acids in the mature polypeptide of SEQ ID NO: 5;
(e) a polypeptide encoded by a polynucleotide having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide encoding sequence of SEQ ID NO: 24; and
(f) a fragment of the polypeptide of (a), (b), (c), (d), or (e) that has thioredoxin activity activity.

89. A composition comprising the polypeptide of claim 88.

90. An isolated or purified polynucleotide encoding the polypeptide of 88.

91. A nucleic acid construct or expression vector comprising the polynucleotide of claim 90, wherein the polynucleotide is operably linked to one or more heterologous control sequences that direct the production of the polypeptide in an expression host.

92. A recombinant host cell comprising the polynucleotide of claim 90 operably linked to one or more heterologous control sequences that direct the production of the polypeptide.

93. A method of producing a polypeptide having thioredoxin peptide activity, comprising cultivating the recombinant host cell of claim 92 under conditions conducive for production of the polypeptide, and optionally recovering the polypeptide.
